(19) United States
(12) Patent Application Publication
(10) Pub. No.: US 2004/0072220 A1
(43) Pub. Date: Apr. 15, 2004

(54) Neurotonin and use thereof

(76) Inventor: Toshihiro Nakajima, Kanagawa (JP)

Correspondence Address: Kent E Baldauf, 700 Koppers Building, 436 Seventh Avenue, Pittsburgh, PA, 15219-1818, US

(21) Appl. No.: 10/467,582

(22) PCT Filed: Feb. 4, 2002
(86) PCT No.: PCT/JP02/00885

(30) Foreign Application Priority Data
Feb. 9, 2001 (JP) 2001-34217

(51) Int. Cl. C12Q 1/68; C07H 21/04; C07K 14/47; C12P 21/02; C12N 5/06

(52) U.S. Cl. 435/6; 435/69.1; 435/320.1; 435/325; 530/350; 536/23.5

(57) ABSTRACT

To specify a substance causing an overexcitation of an Isaacs syndrome and to establish an effective diagnosing and treating method, an immunoscreening method was applied to a serum obtained from an Isaacs syndrome patient. A specific cDNA was separated from a cDNA library derived from a nerve cell. The nucleotide sequence was elucidated and a novel protein, i.e., a neurotonin was obtained by cloning based thereon. The novel protein of which amino acid sequence was also determined is regarded as a key substance of the Isaacs syndrome and is useful for the studies of peripheral nerve lesions and a clinical application.

### TECHNICAL FIELD

The present invention relates to neurotonin and utilization thereof. More specifically, the present invention relates to a neurotonin, a cDNA for encoding the neurotonin, a related nucleic acid molecule, a monoclonal antibody to the neurotonin and the like, and utilization thereof.

### BACKGROUND ART

Peripheral nerve lesions result from the defect of an excitation transmission of a nerve in the neuromuscular junction terminals of a motor nerve and often cause a myopathy. For this reason, there have been known various disease conditions, for example, a sensation dysesthesia, motion disorders, autonomic nerve lesions and the like. The cause of the diseases is also diversified and, in detail, is not known in many cases. An Isaacs syndrome has been known as a neuropathy and myopathy causing the VGKC (Voltage gated potassium channel) abnormality of nerve terminals and the continuous discharge of acetylcholine from the nerve terminals, and has also been referred to as a syndrome of continuous muscle-fiber activity. As a clinical feature, there have been observed symptoms, for example, relaxation difficulties caused after muscular contraction such as the muscle rigidity of limbs, dolorous muscular convulsion and limited finger abduction, dysbasia, hyperhidrosis and the like. Although the mechanism of the symptoms is not known, the presence of a complication of a thymoma and a case in which a blood contains an immune complex are observed. Consequently, the presence of an autoimmune disease can also be supposed.

Under actual circumstances, there has not been a specific and effective method of treating peripheral nerve lesions including the Isaacs syndrome, and the treatment is limited to a countermeasure therapy, for example, a treating method using a plasma exchange, administration of vitamin B₁₂ and the like. Since the arrival of a real aging society is close at hand, there has been ardently desired a method of effectively carrying out a treatment for peripheral multiple neurosis to be a complication of diabetes, the whole painful numbness of a muscle and the like in addition to the peripheral nerve lesions.

The present inventor grasped the fact that a cDNA isolated from an Isaacs syndrome patient encodes a certain specific protein and the protein is increased in the blood of the patient. As a result of intensive studies, the present inventor identified the cDNA and the protein and ascertained that they are factors related to the disease conditions, and determined their sequence structure and thus completed the present invention related to a diagnosis and treatment utilizing the nucleic acid molecules and protein. The DNA molecule and the protein according to the present invention provide various substances and methods which contribute to basic studies related to peripheral nerve lesions and clinical applications.

### OBJECT OF THE INVENTION

It is an object of the present invention, on the basis of the structure and function of neurotonin or cDNA coding therefor, to provide a nucleic acid molecule, a monoclonal antibody to a neurotonin, clinical testing chemicals, a remedy, a novel vector and a blood filtering material conjugating an antineurotonin antibody, a culture medium, transgenic animals, animals for screening and the like by utilizing them.

### SUMMARY OF THE INVENTION

The present invention provides a peripheral nerve cell protein having the following properties (which will be hereinafter referred to as a “neurotonin”),

wherein the protein

a) is found in peripheral nerve cells of Isaacs syndrome patients or other patients having peripheral nerve lesions,

b) reacts with an antibody found in a blood of the patients,

c) causes a similar symptom to an Isaacs syndrome group with an immunization to a rabbit, and

d) has a molecular weight of approximately 66 kDa according to SDS-PAGE.

The DNA according to the present invention is a cDNA encoding the neurotonin.

Furthermore, the DNA according to the present invention is a DNA encoding the neurotonin or including all nucleotide sequences shown in FIG. 1 or 2.

The present invention also includes a nucleic acid molecule encoding a neurotonin, including at least one nucleotide sequence substantially corresponding to a whole or part of the nucleotide sequences shown in FIG. 1 or 2, or including a sequence which is substantially homologous with any of the sequences or is hybridized with any of the sequences.

The DNA according to the present invention also includes a DNA encoding a mouse neurotonin or containing all nucleotide sequences shown in FIG. 3 or 4. Moreover, a mouse cDNA encoding the mouse neurotonin also belongs to the present invention.

Moreover, the present invention also includes a nucleic acid molecule encoding a mouse neurotonin, including at least one nucleotide sequence substantially corresponding to a whole or part of the nucleotide sequences shown in FIG. 3 or 4, or including a sequence encoding the mouse neurotonin which is substantially homologous with any of the sequences or is hybridized with any of the sequences.

Furthermore, the present invention also includes an antisense RNA or an antisense DNA to the cDNA or any of the nucleic acid molecules, or a ribozyme recognizing and cleaving said cDNA, said nucleic acid molecule or an RNA transcribed from a part thereof.

An expression vector, a cloning vector or a cosmid according to the present invention includes the cDNA or any of said nucleic acid molecules.

A transformant according to the present invention retains the vector or the cosmid.

A prokaryotic cell, a eukaryotic cell or a variant cell according to the present invention contains any of the nucleic acid molecules described above.

A neurotonin according to the present invention has an amino acid sequence shown in FIG. 5 or 6.

A protein according to the present invention has an activity to bind to an antibody found in a blood of the patient and the following feature a) or b).

a) A polypeptide such as a polypeptide having an amino acid sequence shown in FIG. 5 or 6, a polypeptide having an amino acid sequence in which at least one amino acid is deleted, substituted or added and/or inserted in the amino acid sequence shown in FIG. 5 or 6, a synthetic polypeptide including an amino acid sequence constituting an antigenic portion of a neurotonin, functionally equivalent variants thereof, or a fused polypeptide further including the aforesaid amino acid sequences, and

b) a protein encoded by a DNA to be hybridizable with the DNA having a nucleotide sequence shown in any of FIGS. 1 to 4.

Moreover, a protein according to the present invention can bind to a “14-3-3” protein existing in a peripheral nerve cell and has the following feature a) or b).

a) A polypeptide such as a polypeptide having an amino acid sequence shown in FIG. 5 or 6, a polypeptide having an amino acid sequence in which at least one amino acid is deleted, substituted or added and/or inserted in the amino acid sequence shown in FIG. 5 or 6, a synthetic polypeptide including an amino-acid sequence constituting an antigenic portion of a neurotonin, functionally equivalent variants thereof, or a fused polypeptide further including the aforesaid amino acid sequences, and

b) a protein encoded by a DNA to be hybridizable with the DNA having a nucleotide sequence shown in any of FIGS. 1 to 4.

Furthermore, the present invention includes an immunologically active domain of the protein or a fragment having the domain.

A polypeptide according to the present invention also includes a polypeptide having an amino acid sequence shown in FIG. 7 or 8, a polypeptide having an amino acid sequence in which at least one amino acid is deleted, substituted or added and/or inserted in the amino acid sequence shown in FIG. 7 or 8, a synthetic polypeptide including an amino acid sequence constituting an antigenic portion of a neurotonin, functionally equivalent variants thereof or a fused polypeptide further including the aforesaid amino acid sequences.

A reagent according to the present invention comprising any of the aforesaid proteins for an immunological analysis serves to assay an antibody recognizing the protein.

Moreover, said reagent for an immunological analysis is used to assess a diagnosis or treatment effect of an Isaacs syndrome or other peripheral nerve lesions.

A reagent according to the present invention comprising an antibody to react to any of the aforesaid proteins for an immunological analysis serves to analyze the protein.

Moreover, the reagent for an immunological analysis is used to assess a diagnosis or treatment effect of an Isaacs syndrome or other peripheral nerve lesions.

In the reagent for an immunological analysis, furthermore, any of the proteins to be analyzed is present in peripheral nerve cells.

The present invention provides a neurotonin detecting method of analyzing an antibody which is present in a body fluid of a subject and reacts with any of the proteins.

A method of screening a ligand bind to the protein according to the present invention comprises the following steps:

a) causing a candidate compound of the ligand to come in contact with the protein described above; and

b) selecting a candidate compound having a binding activity to the protein.

The present invention provides an assay method of measuring any of the proteins on the basis of an affinity of an antiligand and the ligand obtainable by the screening method by using, as the antiligand, the protein.

A screening method according to the present invention for a compound inhibiting a binding of any of the proteins to the ligand and comprises the following steps:

a) causing the protein to come in contact with the ligand under the presence of a candidate compound; and

b) selecting a candidate compound having an activity to inhibit the binding of the protein to the ligand.

A compound according to the present invention also includes a compound which can be obtained by the screening method.

A neurotonin inhibitor according to the present invention comprises a compound which can be obtained by the screening method.

A medicine for treating peripheral nerve lesions according to the present invention comprises a compound which can be obtained by the screening method.

An antibody according to the present invention is an antibody capable of binding to any of the proteins.

The antibody includes a monoclonal antibody. Another monoclonal antibody according to the present invention is a monoclonal antibody against an antibody causing peripheral nerve lesions.

A method according to the present invention includes a method of detecting or separating a cell expressing any of the aforesaid proteins by setting, as a marker, the protein or an expression of a gene encoding the protein. This method includes the case in which the cell is a peripheral nerve cell.

A reagent for detecting or separating a cell expressing any of the proteins according to the present invention comprises the antibody described above.

A reagent according to the present invention comprises a reagent containing a nucleic acid molecule having a nucleotide sequence hybridizable with the DNA or a part thereof, or an RNA transcribed therefrom and detecting the DNA or the RNA.

A transgenic non-human vertebrate according to the present invention is a transgenic non-human vertebrate in which an expression of the DNA is modified or inducible the modification.

The animal is e.g. a peripheral nerve lesion model animal.

The knockout non-human vertebrate according to the present invention is the non-human vertebrate in which an expression of the intrinsic DNA is suppressed.

The knockout non-human vertebrate also includes a non-human vertebrate in which another gene is knocked out.

A cell according to the present invention includes a cell derived from any of the aforesaid non-human vertebrates.

A screening method according to the present invention also includes a method of screening a compound to increase or reduce an activity of an intrinsic promoter of the DNA shown in FIGS. 1 to 4, comprising the steps of:

a) detecting an expression of a gene bound to a downstream region of the intrinsic promoter of the DNA shown in FIGS. 1 to 4 under the presence of a compound to be tested; and

b) selecting the compound to increase or reduce the expression.

Moreover, the screening method of the present invention includes a method of screening a compound to increase or reduce an activity of an intrinsic promoter of the DNA shown in FIGS. 1 to 4, comprises the steps of:

a) applying a compound to be tested to the non-human vertebrate or a cell derived from the animal; and

b) selecting the compound to increase or reduce an expression of a gene which is knocked in.

An animal immunized with a neurotonin according to the present invention is an animal immunized with the neurotonin which is to be used for studies of an Isaacs syndrome or other peripheral nerve lesions and for screening of a substance having an improvement action of the Isaacs syndrome or other peripheral nerve lesions.

According to the present invention, a rabbit is immunized with a neurotonin for screening a protein or a non-protein substance which improves a suppressed VGKC function, a signal transduction disorder in a nerve terminal or a painful muscular convulsion.

The present invention provides a diagnosing marker for detecting a specific conjugation with the monoclonal antibody by using, as a marker, a neurotonin, a related protein thereto or other antibodies causing peripheral nerve lesions, thereby measuring a level of a neurotonin, a related protein thereto or an antibody causing the peripheral nerve lesions in a sample test body obtained from a subject to thereby diagnose an Isaacs syndrome or other peripheral nerve lesions.

The present invention provides an examination method of offering data for diagnosing an Isaacs syndrome or other peripheral nerve lesions, wherein a level of a neurotonin or an antibody causing peripheral nerve lesions in a sample obtained from a subject is measured.

The present invention provides a blood filtering material for use in eliminating a numbness caused by the presence of a neurotonin or an antibody causative peripheral nerve lesions to carry out a treatment which allows a neurotonin antibody or an antibody causing the peripheral nerve lesions in a blood to bind to the monoclonal antibody fixed onto the filtering material through a dialytic filtration of the blood, thereby reducing the neurotonin antibody or the antibody causative the peripheral nerve lesions in a body.

The present invention provides a vaccine composition for stimulating an immune response to an Isaacs syndrome or other peripheral nerve lesions in a human being or other animals, the composition serving to stimulate an immune response against at least one polypeptide described above which is contained together with a pharmaceutically acceptable carrier in the composition, or an immune response against a polypeptide encoded by a inserted nucleic acid molecule in a virus cell or the host cell which is contained in the composition.

The present invention provides a medium for a cell culture comprising the monoclonal antibody according to claim 31 or 32 for in vitro reproducing a cell to be a peripheral nerve by removing an antibody causative peripheral nerve lesions through an incubation with peripheral nerve and muscle system cells separated from a patient having peripheral nerve lesions, to thereby cause a specific binding of the causative antibody to the monoclonal antibody.

### BRIEF DESCRIPTION OF THE DRAWINGS

FIG. 1 shows a nucleotide sequence of a human neurotonin coding cDNA (short).

FIG. 2 shows a nucleotide sequence of a human neurotonin coding cDNA (long).

FIG. 3 shows a nucleotide sequence of a mouse neurotonin coding cDNA (short).

FIG. 4 shows a nucleotide sequence of a mouse neurotonin coding cDNA (long)

FIG. 5 shows an amino acid sequence of a human neurotonin (short).

FIG. 6 shows an amino acid sequence of a human neurotonin (long).

FIG. 7 shows an amino acid sequence of a mouse neurotonin (short)

FIG. 8 shows an amino acid sequence of a mouse neurotonin (long)

FIG. 9 is a photograph showing the detection of a neurotonin and the estimation of its molecular weight by Western blotting, also illustrating the result of a rat (PC-12) together with an NB-1 nerve cell (left side) 66 KDa corresponds to the neurotonin (long) and 44 KDa corresponds to the neurotonin (short)

FIG. 10 is a photograph showing the induction of peripheral nerve lesions caused by immunizing a rabbit with a human neurotonin. A indicates a normal rabbit holding a natural lie-down position. B indicates a rabbit suffering from the peripheral nerve lesions induced after the immunization which has a numb hind limb and presents dysbasia.

FIG. 11 is a photograph showing the muscular tissue abnormality of a rabbit which is caused by an immunization with the neurotonin. A indicates the muscular tissue of a normal rabbit and B indicates the muscular tissue of the rabbit having the peripheral nerve lesions. A result obtained by an HE stain is shown in a 100-fold magnification.

FIG. 12 is a photograph showing a result obtained by detecting an antibody raised against a neurotonin in a human serum by using the human neurotonin. Horseraddish peroxidase conjugated—antihuman immunoglobulin M (anti-human IgM-HRP) was used as a secondary antibody. A indicates the serum of a patient M having the peripheral nerve lesions, B indicates the serum of a patient A having the peripheral nerve lesions, and C indicates the serum of a normal human.

### DETAILED DESCRIPTION OF THE INVENTION

The present invention provides a peripheral nerve cell protein having the following properties, wherein the protein

a) is found in peripheral nerve cells of Isaacs syndrome patients or other patients having peripheral nerve lesions,

b) reacts with an antibody found in a blood of the patients,

c) causes a similar symptom to an Isaacs syndrome group with an immunization to a rabbit, and

d) has a molecular weight of approximately 66 kDa according to SDS-PAGE.

The present invention will be described below in detail in order of a cDNA encoding a neurotonin, a related nucleic acid molecule, the neurotonin, the physiological function of the neurotonin, a related protein, a medical material and an application to animals.

In this specification, the “peripheral nerve lesions” indicate the disorders of peripheral nerve except an Isaacs syndrome. In the text, this term will be used in a broad sense including the Isaacs syndrome if necessary.

“VGKC” is an abbreviation of voltage gated potassium channel, which implies an electric-potential dependent potassium channel.

In “a neurotonin and a related protein thereto” and “a neurotonin or a related protein thereto”, the “related protein thereto” also includes an antibody against neurotonin.

cDNA Encoding Neurotonin

A cDNA encoding a neurotonin may be prepared by the application of an immunoscreening method using the serum of an Isaacs syndrome patient to a cDNA library corresponding to a human genome. More specifically, the cDNA library derived from a nerve such as an NB-1 cell is created by separating mRNAs through a conventional method and incorporating the mRNAs into a λ vector or the like by a well-known technique, and allowing a reverse transcriptase to act on the mRNA mixture. The serum containing the anti-neurotonin antibody is applied for the cDNA library so that a clone containing a cDNA expressing a neurotonin is identified

In a process in which the present inventor carried out the immunoscreening by using the sera from Isaacs syndrome patients to obtain a cDNA encoding a novel protein as described above, two types of cDNA having different chain lengths were found. The long cDNA encoding the protein will be referred to as a “long cDNA” while the short cDNA encoding a protein of 386 amino acids which was first obtained will be referred to as a “short cDNA”.

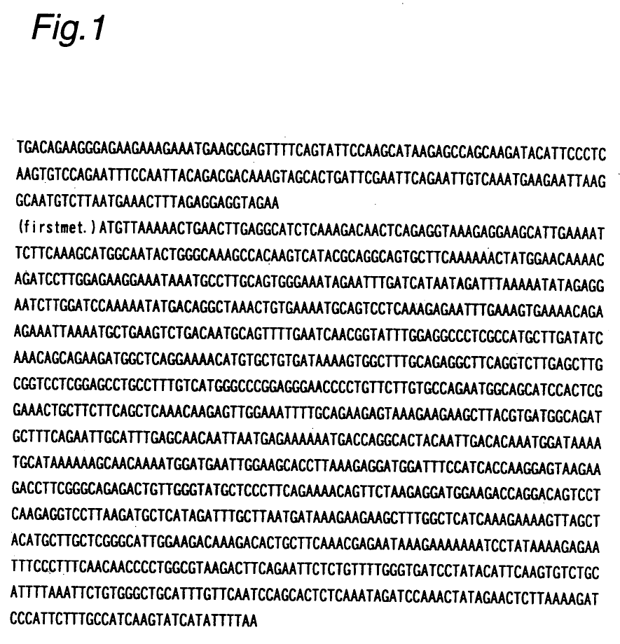

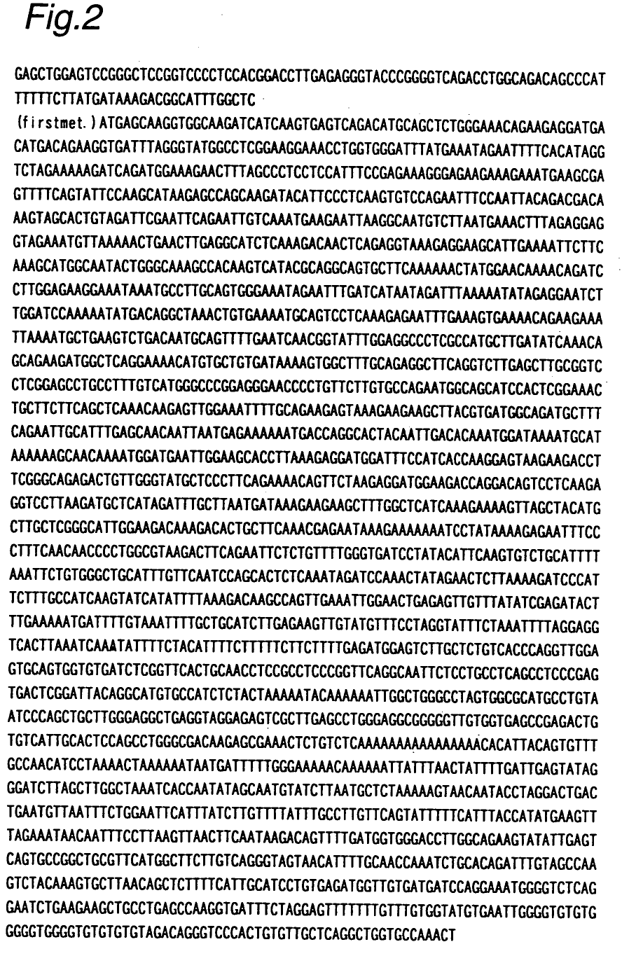

FIGS. 1 and 2 show a nucleotide sequence of the “short cDNA” and the “long cDNA” for the cDNA encoding the neurotonin.

The DNA according to the present invention encodes the neurotonin or includes the whole nucleotide sequence shown in FIG. 1 or 2. The DNA includes a DNA of a genome structural gene carrying genetic information before splicing as well as a DNA containing initiation and termination codons.

Furthermore, a nucleic acid molecule encoding the neurotonin, including at least one nucleotide sequence substantially corresponding to a whole or part of the nucleotide sequence shown in FIG. 1 or 2, or including a sequence which is substantially homologous with any of the sequences or hybridizable with any of the sequences also belongs to the nucleic acids of the present invention. The nucleic acid molecule according to the present invention may be a single or double stranded DNA, cDNA or RNA.

The “substantially homologous” sequence includes a sequence having approximately 50% or more, for example, 60% or more of a sequence identity, a sequence of functionally equivalent allelic variants, and a related sequence modified by the substitution, addition and/or deletion of at least one nucleotide. Similarly, “functionally equivalent” implies a similar correspondence to a polypeptide indicative of the same functionality as that of the neurotonin.

A nucleic acid molecule hybridizable with the sequences shown in FIGS. 1 and 2, or the substantially homologous sequence or functionally equivalent sequence which is defined as described above is also included within the scope of the present invention. “Hybridize” used herein is defined as a sequence being bound under non-stringent condition (6×SSC, 1% SDS, 10% Dextran, 100 g/ml salmon sperm DNA, a room temperature) and washed away under a low stringent condition (2×SSC, a room temperature, more preferably 2×SSC, 30° C.) or a higher stringent condition, for example, 2×SSC, 65° C. SSC is an abbreviation of Standard Saline Citrate and implies 0.15M NaCl and 0.015M sodium citrate pH 7.2.

By utilizing a well-known method of genetic engineering technology, it is also possible to create a nucleic acid molecule including at least one nucleotide sequence encoding a polypeptide of an antibody against the neurotonin and incorporating a region encoding at least one antigenic determinant from the neurotonin encoding sequence shown in FIG. 1 or 2. Such a nucleic acid molecule is also included in the present invention.

In order to detect a structural gene encoding the neurotonin and a DNA derived therefrom, a cDNA derived therefrom, or a DNA comprising at least one nucleotide sequence substantially corresponding to a whole or part of the nucleotide sequence shown in FIG. 1 or 2, a sequence which is substantially homologous with or functionally equivalent to any of the sequences described above, or a sequence hybridizable with any of the sequences and an RNA transcribed from the DNA, a reagent containing these nucleic acid molecules is preferably used based on a principle utilizing a hybridization with their DNA chain or RNA chain. Furthermore, these nucleic acid molecules can be also employed as hybridization probes for searching a PCR primer or a homologous sequence portion.

Conventionally, the probe can be labeled with a radioisotope, more preferably, a nonradioisotope such as a fluorescent dye or chemiluminescence by a 5′ terminal label method, a nick translation method, a random primer met-hod or the like. A reagent for detecting the DNA or the RNA transcribed therefrom is preferably utilized as a probe for a hybridization in an in situ hybridization, a southern hybridization, a northern hybridization, a plaque hybridization, a colony hybridization or the like.

Such a probe is also used preferably in searching for a target clone from a genome library or a cDNA library by the hybridization on a nucleic acid level.

Neurotonin

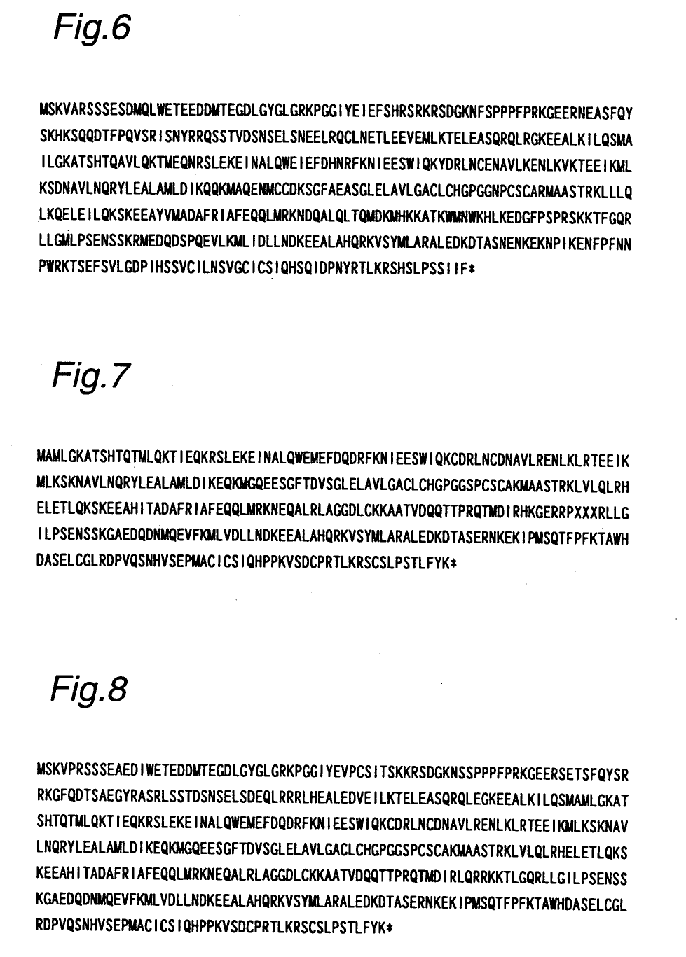

The neurotonin according to the present invention is a protein having an amino acid sequence shown in FIG. 5 or 6, or a protein encoded by the cDNA, a gene corresponding to the cDNA or a DNA hybridizable with any of the nucleotide sequences described above. The neurotonin has such an activity as to be bound to an antibody found in the blood of the above-described patient, and furthermore, can be conjugated to a “14-3-3 protein” in a peripheral nerve cell.

The neurotonin may be obtained by immunoscreening utilizing the sera of an Isaacs syndrome patient or of other patients having peripheral nerve lesions. Alternatively, if the cDNA is available, the neurotonin can also be prepared through the transcription and translation of the cDNA by utilizing a well-known recombinant DNA technology.

Furthermore, the protein according to the present invention is extended to a polypeptide in such a chemical structure as to be a polypeptide having an amino acid sequence shown in FIG. 5 or 6, a polypeptide having an amino acid sequence in which at least one amino acid is deleted, substituted or added and/or inserted in the amino acid sequence shown in FIG.

or 6, a synthetic polypeptide containing an amino acid sequence constituting the antigenic moiety of the neurotonin, a polypeptide of their variant polypeptides which is functionally equivalent or a polypeptide of a fused polypeptide type further containing their amino acid sequences, and these polypeptides are also included in the present invention. Moreover, the protein according to the present invention may be obtained by a partial or whole artificial synthesis or by modification of the protein obtained from a biological source in addition to the protein of the biological source. The protein of the biological source according to the present invention also includes a protein manufactured based on the recombinant DNA technology.

The “polypeptide” used herein includes both a protein having a full length sequence and a polypeptide having a shorter sequence than the protein. Consequently, an immunologically active domain of the neurotonin or a fragment having the domain is also included in the present invention. “Immunologically active” implies that the domain includes an epitope against an antibody directed to the protein and has an antigenicity. Accordingly, a fragment including such a domain retains the antigenicity. Similarly, the synthetic polypeptide comprising an amino acid sequence constituting the antigenic portion of the neurotonin is also included in the protein according to the present invention.

The term of “functionally equivalent” used above in relation to the amino acid sequence of the polypeptide is referred to the situation where an amino acid sequence is modified by the deletion, substitution, addition and/or insertion of at least one amino acid, or an amino acid sequence is otherwise obtained by chemically modifying the side group of an amino acid residue through phosphorylation or dephosphorylation, glucosylation, deglucosylation or the like, for example, and a substantially equivalent action or activity to that of the neurotonin is retained irrespective of the altered amino acid sequence. Such a functionally equivalent variant is generated as a natural biological mutation in some cases or can also be produced by utilizing a well-known technique. For example, a functionally equivalent recombinant polypeptide can be generated by using a well-known technique such as the mutagenesis at a specific site, the mutagenes is at an unspecific site, the enzymic cleavage and/or ligation of an amino acid or the like.

The protein, that is, the neurotonin and related proteins thereto can be used for analyzing an antibody to recognize and bind to the proteins. For this purpose, a reagent for an immunological analysis includes any of the proteins described above and can detect the antibody in a specimen through an antigen-antibody reaction and the measurement of detecting any entity linked to the reaction. As a detecting entity, it is possible to properly exploit well-known detecting means such as an immuno-antibody method, a method utilizing a radioactivity or the like in addition to various spectroscopic methods including fluorescence or chemiluminescence.

Since such a reagent for the immunological analysis serves to detect and analyze a specific complexing of the antibody to be a disease marker with the neurotonin or a related protein thereto, it can also be utilized for a diagnosis. More specifically, it is also possible to utilize the reagent for diagnosing the Isaacs syndrome or other peripheral nerve lesions or to assess a curative effect by measuring the level of the specific antibody against the neurotonin in a specimen from a subject or an antibody responsible for the peripheral nerve lesions.

As another embodiment, it is possible to use the reagent in order to detect a fused cell producing an antibody, that is, to efficiently detect a fused cell producing a specific target antibody from a large number of fused cells in the manufacture of a monoclonal antibody described hereinafter. For example, a protein such as the neurotonin to be an antigen is fixed onto a solid-phase support and thereby an antibody in a fused cell supernatant to be bound thereto is detected.

A substance capable of binding to the neurotonin or related proteins thereto or a compound having a ligand which can be bound thereto is useful for a substance to control the function of the neurotonin, including the antibody or a substance which can be utilized for an analysis. Furthermore, it is also possible to employ a compound having a suppressing function for the production of an anti-neurotonin antibody, a compound likely affecting the action of the anti-neurotonin antibody, or a compound capable of blocking the coupling of the antibody to the neurotonin. These compounds may exhibit some pharmacological activity in the Isaacs syndrome or other peripheral nerve lesions.

Preferably, a screening method for selecting such substances includes the following steps:

a) a step of allowing the candidate compound of a ligand to come in contact with the neurotonin or related proteins thereto; and

b) a step of selecting a candidate compound having a binding activity to the protein.

For analyzing the protein, it can be measured through a specific binding to a ligand to be obtainable by the screening method using the protein as an antiligand.

Furthermore, it is preferable that a method of screening a compound inhibiting the binding of said protein to the ligand thereof should comprise the following steps:

a) a step of allowing the neurotonin or related proteins thereto to come in contact with the ligand under the presence of a candidate compound; and

b) a step of selecting a candidate compound having an activity to inhibit the binding of the protein to the ligand thereof.

The ligand or compound which can be obtained by the screening method may be an inhibitor for the neurotonin or related proteins thereto. By utilizing the binding of the neurotonin or related proteins thereto, accordingly, the compound obtained by the screening may be used as a medicine for a treatment of the peripheral nerve lesions, including the Isaacs syndrome.

cDNA Encoding Mouse Neurotonin

It is possible to isolate a cDNA encoding a mouse neurotonin from a mouse cDNA library through hybridization cloning. For this purpose, it is also possible to use a full length sequence of the neurotonin cDNA as a probe, thereby screening a plaque containing a positive clone.

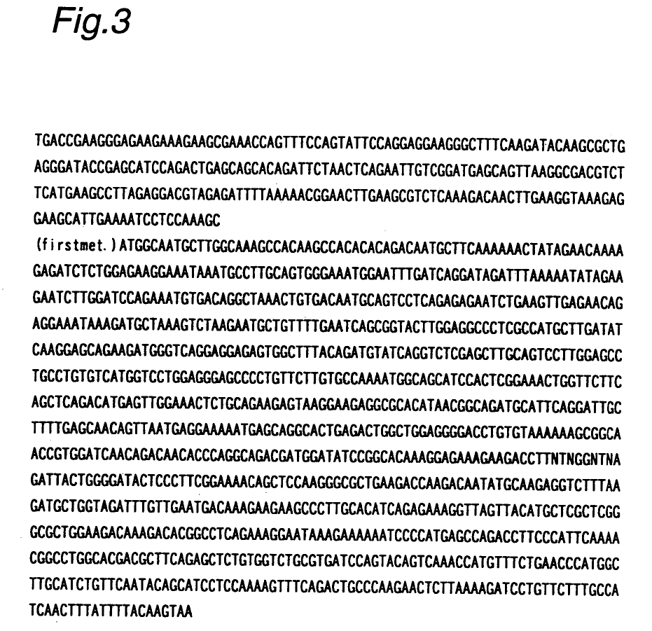

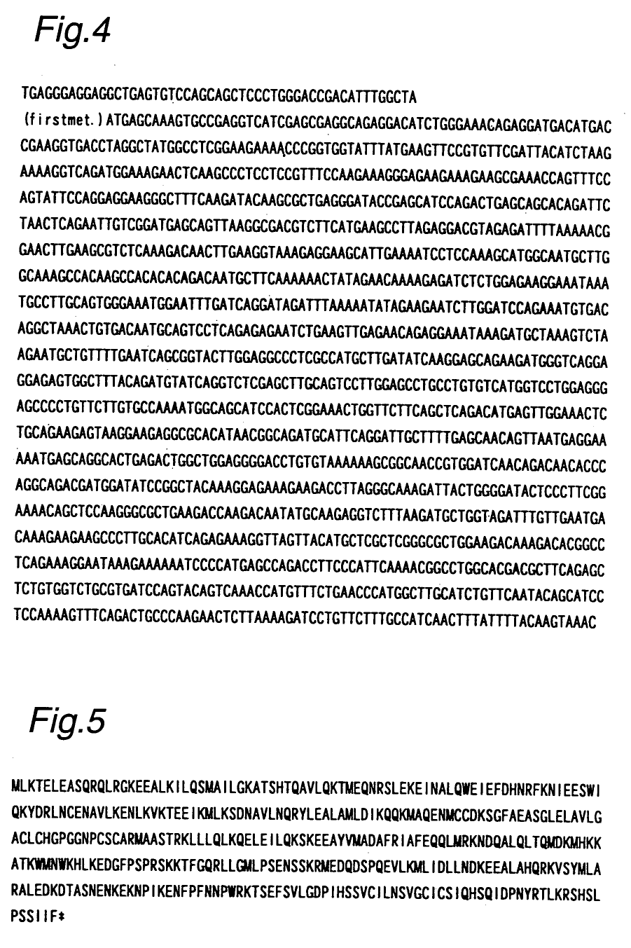

FIGS. 3 and 4 show a nucleotide sequence of a cDNA encoding the neurotonin of a mouse which is thus isolated and sequenced.

A DNA according to the present invention includes a DNA encoding the mouse neurotonin or a DNA containing whole the nucleotide sequence shown in FIG. 3 or 4. Furthermore, the present invention also includes a nucleic acid molecule which encodes the mouse neurotonin, contains at least one nucleotide sequence substantially corresponding to a whole or part of the nucleotide sequence shown in FIG. 3 or 4, is substantially homologous with any of the sequences or contains a sequence hybridizable with any of the sequences. The nucleic acid according to the present invention may be a single or double stranded DNA, cDNA or RNA.

The “substantially homologous” sequence includes a sequence having approximately 50% or more, for example, 60% or more of a sequence identity, a functionally equivalent allelic variant, and a related sequence thereto modified by the substitution, addition and/or deletion of at least one nucleotide. “Functionally equivalent” implies to have a sequence encoding a polypeptide indicative of the same functionality as that of the neurotonin.

A nucleic acid molecule hybridizable with the sequences shown in FIGS. 1 and 2, or the substantially homologous or functionally equivalent sequence as defined above is also included in the scope of the present invention. “Hybridize” used herein is defined as described above.

By utilizing a well-known genetic engineering technology, similarly, it is possible to create a nucleic acid molecule including at least one nucleotide sequence encoding a polypeptide of an antibody against the neurotonin and incorporating a region encoding at least one antigenic determinant from the neurotonin encoding sequence shown in FIG. 3 or 4.

The above-described matters, utilization and the like for the DNA encoding the human neurotonin or the nucleic acid molecule exactly apply to a nucleic acid encoding a mouse neurotonin and a related protein thereto.

Mouse Neurotonin

A mouse neurotonin according to the present invention is a polypeptide having an amino acid sequence shown in FIG. 7 or 8. In addition to the mouse neurotonin, a polypeptide of the present invention, furthermore, is extended to a polypeptide in such a form as to be a polypeptide having an amino acid sequence in which at least one amino acid is deleted, substituted or added and/or inserted in the amino acid sequence shown in FIG. 7 or 8, a synthetic polypeptide containing an amino acid sequence constituting the antigenic portion of the neurotonin, their variant which is functionally equivalent or a fused polypeptide further containing their amino acid sequences. The “polypeptide” and “functionally equivalent” used herein are defined as described above.

The above-described matters, utilization and the like for the human neurotonin exactly apply to a mouse neurotonin and a related protein thereto.

Physiological Function of Neurotonin and Neurotonin Gene

A human genome contains a structural gene having information about the amino acid sequence of a neurotonin which is a protein newly found by the inventor through an immunoscreening method utilizing a relevant antibody present in the serum of an Isaacs syndrome patient. The details of a position, an expression physiology and a control function thereof have not been known. Further elucidation of the problem as to whether the Isaacs syndrome or other peripheral nerve lesions result(s) from the excessive expression of the neurotonin or are (is) caused derivatively by an ectopic expression is awaited. Also the relationship between a neurotonin and an antibody thereto and the peripheral nerve lesions should be resolved.

A human neurotonin is also the protein with which immunization may evoke the symptom of the Isaacs syndrome in the animal other than a human being. It is possible to use any animal which gives an immune response, for example, Primates, sheep, rabbits, rats, mice or the like.

For example, in case of a rabbit, a fused protein with GST is produced by using the neurotonin containing 511 amino acid residues in a total length and the rabbit is immunized with the fused protein to be an antigen. After approximately six months since inoculation, it is observed that peripheral nerve lesions showing a similar symptom to the Isaacs syndrome are induced, for example, a numbness in the hind limbs. In the muscle of the rabbit immunized with the neurotonin, the size of muscular cells is nonuniform differently from a normal muscular tissue, and the denaturation of a nerve is also observed.

In the Isaacs syndrome patient, the expression of the neurotonin gene is enhanced so that the blood level of an antibody to the neurotonin encoded thereby is raised. The cause of the expression of the gene, the physiological function of the neurotonin, and the relationship with the disease condition of the Isaacs syndrome are unknown. Since an antibody production against the neurotonin is also increased, there are also caused likely peripheral nerve lesions based on the outcome of an auto-immunity disease. Together with these observations or the results of experiments, it is acknowledged that the newly found and identified neurotonin is a protein closely associated with the Isaacs syndrome or other peripheral nerve lesions.

The cDNA of a mouse neurotonin is homologous by 85% or more with the nucleotide sequence of a human cDNA corresponding thereto. In an amino acid sequence level derived from the mouse neurotonin cDNA, another factor on homology is not found and its function is also unknown. The mouse neurotonin gene also functions in the same manner as the human neurotonin gene. It can be supposed that similar peripheral nerve lesions to the Isaacs syndrome are induced, for example, a hind limb has a numbness if the mouse is immunized with the human neurotonin.

Referring to the expression of the gene, a compound to increase or reduce the activity of the intrinsic promoter of the DNA may be screened by a method comprising the steps of:

a) detecting the expression of a gene bound to the downstream region of the intrinsic promoter of the DNA under the presence of a compound to be tested; and

b) selecting the compound to increase or reduce the expression.

The nucleotide sequence of the human neurotonin cDNA is converted into a corresponding amino acid sequence to carry out a motif search. Consequently, it is clear that a main motif is absent but a site to be modified by a lipid is present on the C terminal side. Based on the fact that the hind limb of the rabbit had a numbness through the immunization using the portion on the C terminal side of the neurotonin and that more antibodies to the neurotonin existing in the serum of a patient complaining of the numbness are found to be those for the C terminal region of the neurotonin, the neurotonin is a membrane protein and at least the C terminal portion thereof functions in a membrane.

From the present inventor's study, it has been elucidated that the C terminal portion of the neurotonin is bound to a “14-3-3 protein”. The “14-3-3 protein” is present in a cell and can recognize proteins having phosphorylated serins residues, and its role as various modulators for a signal transduction is assumed. Accordingly, a protein or a nonprotein substance capable of binding to or interacting with the “14-3-3 protein” in place of the neurotonin or competitively may exert a similar influence to the neurotonin on the “14-3-3 protein”. It is likely that such a protein or nonprotein substance may exhibit some pharmacological action in the Isaacs syndrome or other peripheral nerve lesions.

It has been reported that an antibody to VGKC is detected in the serum from some of patients having the Isaacs syndrome or other peripheral nerve lesions. For these disease conditions, the functional abnormality of VGKC is suggested to be responsible. Furthermore, it is apparent from the study of the present inventor that various antibodies against the neurotonin are also present in the sera of the patients, and particularly, a large number of patients have the antibody against the C terminal region of the neurotonin. Similarly, these antibodies are also assumed to be involved in the symptom in relation to the physiological function of the neurotonin. For this reason, an antibody which can bind to these antibodies, a compound capable of exerting a suppressing action on the antibody production, a compound which might influence the action of the antibody, a compound capable of blocking the binding of the antibody to the neurotonin or the like may exhibit some pharmacological action in the Isaacs syndrome or other peripheral nerve lesions.

Related Nucleic Acid Molecule

An antisense RNA and an antisense DNA incorporating the neurotonin gene, an mRNA, a cDNA or a nucleic acid molecule related thereto according to the present invention by a recombinant DNA technology as well as, a ribozyme, a cosmid and the like are also included in the scope of the present invention. If necessary, the recombinant DNA and the nucleic acid molecule can also be utilized in various gene cloning operations applicable in the establishment of gene diagnosing methods and gene therapy and the development of drugs.

Antisense RNA and Antisense DNA

Since a double stranded RNA or DNA produced by a hybridization of an mRNA chain carrying the translation information of the neurotonin with an antisense chain complementary thereto, or an antisense RNA or antisense DNA against the cDNA or the nucleic acid molecules above described has an ability of inhibiting a translation through a hybridization with the complementary mRNA thereto and suppressing thereby the expression of a corresponding gene, it may be applied to an antisense medical treatment and the creation of a knockout mouse.

In the antisense medical therapy, the antisense RNA and the antisense DNA are utilized in the clarification of the mechanism of a disease onset on a gene level, the development of a treating method and the like. In order to control the expression of the neurotonin gene, moreover, it is possible to utilize the RNA and the DNA. More particularly in order to suppress the expression of a target gene, the antisense RNA may be injected into a cell, or alternatively the DNA to create the antisense RNA may be introduced therein.

Ribozyme

The present invention also includes, among the ribozymes whose enzymatic activity has been demonstrated, the ribozyme designed to specifically cleave an optional nucleotide sequence of the RNA for the neurotonin, a related RNA transcribed from the above-mentioned cDNA or the nucleic acid molecule, or a related RNA transcribed from a part thereof and produced by a chemical synthesis, or an oligo DNA having the nucleotide sequence of the enzyme active site and an RNA-cleaving activity.

Cosmid

Since a circular DNA incorporating the neurotonin gene, a DNA fragment thereof or a related nucleic acid thereto as well as necessary elements, such as, an origin of replication derived from a plasmid, a cos site of a λ bacteriophage are replicable in the same manner as the plasmid, it can be utilized as a vector. Such an artificial nucleic acid particle may incorporate therein a comparatively large DNA fragment. Therefore, for the purpose of cloning, it is possible to efficiently introduce the artificial nucleic acid particle into a bacterium such as Escherichia coli. Moreover, the artificial nucleic acid particle is also suitable for the preparation of a gene library.

Expression Vector and Cloning Vector

A microorganism cell such as a prokaryotic cell or a eukaryotic cell which contains the transformed nucleic acid may be used for the expression in a large amount of a protein of interest.

The nucleotide sequence encoding the neurotonin according to the present invention can be so cloned as to bring about its expression by properly utilizing a group of well-known techniques and expression systems, for example, an expression system in the prokaryotic cell such as Escherichia coli or a Bacillus subtilis, an expression system in the eukaryotic cell such as a yeast or a transformed mammalian cell, an expression system in a transgenic mammals and the like.

It is possible to preferably use a method of preparing the synthetic polypeptide related to the neurotonin, comprising the steps of culturing the eukaryotic cell or prokaryotic cell incorporating the nucleic acid molecule under such a condition that the polypeptide is expressed, and collecting the polypeptide thus produced.

As described above, the scope of the present invention encompasses a cloning vector and an expression vector which contain the nucleic acid molecule or the nucleotide sequence according to the present invention. Such an expression vector incorporates a proper control sequence in which a reading frame and a nucleic acid molecule according to the present invention are fittingly ligated, for example, a translation control element such as start and stop codons, and transcriptional control elements such as a promoter operator region, ribosomal binding sites and an ending stop sequence.

Examples of the vector according to the present invention include plasmid, bacteriophage, a virus (including a eukaryotic virus) and the like which have been well-known in the recombinant DNA technology field or described in the documents in the art. These vectors may be used in various well-known expression systems. As a preferred viral vector, a baculovirus, an adenovirus, a vaccinia virus and the like can be illustrated.

In order to include an expression of the nucleic acid molecule or the nucleotide sequence according to the present invention by using various well-known techniques, the vector or cosmid maybe inserted into a prokaryotic cell or a eukaryotic cell. Alternatively, it is possible to prepare a transformant holding the vector or cosmid in a germ cell line or a somatic cell in order to create a transgenic animal.

Furthermore, the present invention also includes a eukaryotic or prokaryotic host cell or a transgenic microorganism which has the nucleic acid molecule or polynucleotide molecule according to the present invention and is transformed or transfected. In order to produce these transformants and the like, a well-known transforming technique or transfection technique may be used.

If the neurotonin requires a post-translational modification such as a disulfide bonding, glycosylation or a lipid attachment more or less, a mammal host cell can well reproduce the native morphology of an antigen and an epitope. Therefore, the mammal cell expression system is more preferable. More specifically, the eukaryotic expression system can perform the post-translational modification whereas the Escherichia coli poorly reproduces the native morphology of the antigen and might produce an insoluble protein. Examples of an animal cell which may be preferably used for this purpose include a human or animal fibroblast or amyeloma cell line, for example, an Hela-human cell line, a BHK-infant hamster renal cell, a VERO—monkey renal cell line, an FR3T3-Fisher rat fibroblast, an NIH3T3—mouse fibroblast cell line, a C127I—mouse breast cancer cell line, a CV-1-Africa green monkey renal fibroblast, a 3T6—mouse embryo fibroblast, an L cell—mouse cell line, a CHO—Chinese hamster ovary cell line, an NSONSI—SP2 and other mouse myeloma cell lines, YB2/0 and Y3 mouse myeloma cell lines, rat myeloma cell lines and the like.

Various proper vectors to the mammal cell lines of different classes have been known and generally include a promoter and/or an enhancer which are (is) operatively ligated to a nucleotide sequence encoding a neurotonin or a fragment thereof. Examples of such a suitable promoter include an SV40 initial or late promoter, for example, a PSVL vector, a cytomegalovirus (CMV) promoter, a mouse metallothionine I promoter, a mouse breast cancer virus LTR (long terminal repeat) and the like. The vector carries a suitable marker, e.g., a gene therefor such as a gene of dihydrofolic acid reductase or a glutamine synthetase.

The transfection of a host cell can be carried out by using a standard well-known technology. For this purpose, for example, there has been established a calcium phosphate method, a method using DEAE-dextran or polybrene in place of the calcium phosphate, a protoplast fusion method, an erythrocyte ghost fusion method, a liposome fusion method or a lipofection method, a direct microinjection method, and a transfection method for mammal cell lines using a gene cannon or an electroporation.

In general, a linear DNA may be introduced more easily than a circular DNA.

Related Protein

A synthetic polypeptide related to a neurotonin may be utilized in an antibody production, a vaccine manufacture and the like which will be described below in the same manner as the neurotonin. The synthetic polypeptide can be produced by an expression in a host cell containing a recombinant DNA which is operatively ligated to an expression control sequence and includes the above-described nucleotide sequence, or in a host cell containing a vehicle or a vector carrying the recombinant DNA molecule. Alternatively, the polypeptide may also be expressed by directly injecting a bare DNA molecule of the present invention into the host cell.

The thus expressed synthetic polypeptide may be a fused polypeptide containing a portion exhibiting an immunogenicity of a whole or part of a neurotonin or a further additionally fused polypeptide encoded by the DNA of a recombinant molecule fused thereto. For example, it is desirable to prepare the synthetic neurotonin according to the present invention which is coupled to a protein such as glutathione-S-transferase, phosphatase, β-galactosidase, urease or an HB core (hepatitis B core) antigen or a fused protein thereof containing another polypeptide. Most of such fused proteins are formed by the expression of a recombinant gene in which two coding sequences are bound to a tuned reading frame. Alternatively, the polypeptide can be combined by chemical means in vitro. Such a fused or hybrid derivative is also included in the present invention and the synthetic polypeptide can be manufactured by using chemical methods such as a well-known Merrifield solid-phase synthesizing method.

Antibody Protein

Antibodies to a neurotonin and a related protein thereto are produced in an Isaacs syndrome patient and a patient having other peripheral nerve lesions. Accordingly, various antibodies to the neurotonin are also found to be present in the sera of the patients. Particularly, in the studies of the present inventor, an antibody directed to the C terminal side portion was observed in many patients. These antibodies are also likely involved in a symptom in close relation to the physiological function of the neurotonin. Consequently, an antibody which can be bound to these antineurotonin antibodies may also achieve some pharmacological function in the Isaacs syndrome or the peripheral nerve lesions.

The antibody may be prepared by inoculating animals with a protein to be an antigen and immunizing the animals. Examples of the animals for this purpose include rabbits, sheep, mice, rats and the like which have widely been utilized. However, all the antibodies to be raised are polyclonal which are antibodies resulting from the nonhuman animals, and therefore, are foreign substances for a human.

Among the antibodies, a monoclonal antibody is a uniform antibody capable of specifically binding to a single epitope which is particularly useful and preferable. More specifically, the scope of the present invention includes a monoclonal antibody to a neurotonin, a monoclonal antibody to a VGKC antibody, a monoclonal antibody to an antibody causing the peripheral nerve lesions or other related proteins and the like.

In the preparation of these monoclonal antibodies, it is also possible to utilize a conventional established cell fusing technique for preparing and cloning a hybridoma of a lymphocyte cell obtained from an animal spleen immunized with the neurotonin to be an antigen and a myeloma cell having an HAT sensitivity or to utilize a well-known recombinant DNA technology, resulting in a mass production.

In order to obtain the humanized monoclonal antibody, a chimera or a humanized antibody is produced by the recombinant DNA technology including a fusion of a human antibody cDNA and an antibody cDNA derived from an animal cell obtained from a mouse, a rat or the like. It is also possible to employ a method of producing a human antibody by the utilization of a human derived cell and tumor cell clone, the fusion of a mouse tumor cell and a human cell and the utilization of a transgenic mouse.

Examples of the general application of the antibody include testing chemicals, diagnosing chemicals, a remedy, a reagent for a study, a reagent for an assay, an antibody for immunoscreening and the like, related to a neurotonin, a related protein thereto, and a “14-3-3 protein”. Examples of the utilization embodiment of these antibodies include a reagent for an immunological assay to measure the neurotonin or the related protein thereto which includes an antibody to react with the neurotonin or the related protein thereto or particularly is based on the fact that the antibody is detectably bound to the neurotonin or the like. The “detectable” implies herein that well-known detecting means such as an immune antibody method or a method utilizing a radioactivity as well as various spectroscopic methods may be appropriately applied. Examples of a specific utilization manner include a radioimmunoassay (RIA), an enzyme-labeled antibody measuring method (ELISA) and the like. In the RIA, the antibody labeled by ¹²⁵I or ¹³¹I or the like is fixed to a solid-phase support and a radioactivity thereof is measured. In the ELISA, moreover, the antibody is fixed to the support in the same manner and a secondary antibody attaching alkaliphosphatase, horseradish peroxidase or the like is added to carry out an enzyme reaction, thereby resultingly a color or fluorescence is measured.

As another example of such an embodiment as to utilize the antibody, moreover, there can be proposed a test method of diagnosing the Isaacs syndrome or other peripheral nerve lesions and offering data for assessing the effect of a treatment This test method is characterized in that a neurotonin or related protein thereto in a sample obtained from a subject is examined, or the level of a neurotonin antibody or an antibody causing the peripheral nerve lesions is measured.

Furthermore, a specific binding with the above-described antibody is detected by using, as a marker, the neurotonin or related protein thereto, or the antibody causing the peripheral nerve lesions. Thus, the level of the neurotonin or related protein thereto in the specimen obtained from the subject, or the level of the antibody causing the peripheral nerve lesions is measured. An obtained result is offered to diagnose the Isaacs syndrome or other peripheral nerve lesions or to assess the treatment effect. Referring to these reagents for an analysis, the neurotonin, the related protein thereto or the like to be analyzed is originally present in a nerve cell. By analyzing the antibody present in the body fluid of the subject, however, it is possible to detect the neurotonin or related protein thereto.

By setting, as a clue, the expression of a gene encoding the neurotonin or related protein thereto, it is possible to detect or separate a cell from which said protein is produced. By using an antibody against the neurotonin or related protein thereto, preferably a monoclonal type antibody thereto as detecting means to utilize a flow cytometry method, for example, it is possible to separate or detect a cell expressing these proteins. As a reagent for the detection or the separation, the antibody, more preferably the monoclonal antibody includes an antibody obtained by attaching a fluorescent substance as a label, for example. More specifically, the detection is carried out by following the fluorescent excitation of the antibody bound to the cell and the separation is carried out by means of a cell sorter having an automatic sorting function.

By using this method, it is possible to specifically detect or separate the peripheral nerve cells expressing the neurotonin in a cell population. By using this method, for example, it is possible to detect and separate peripheral nerve cells derived from a transgenic non-human vertebrate or a knockout non-human vertebrate which will be described below. These cells provide a useful material for examining the expression and control of a neurotonin gene.

The present invention also includes a eukaryotic or prokaryotic host cell which comprises the nucleic acid molecule or the nucleotide molecule according to the present invention and is transformed or transfected or a transgenic microorganism. Furthermore, the present invention also includes a fused cell of a B cell producing the monoclonal antibody and a tumor cell. The antibody and the flow cytometry is combined and utilized, and can also be utilized for a separation of the transformant, the fused cell or the like of objects.

Application as Medical Material

The neurotonin and related protein thereto according to the present invention may be utilized for the analysis, the clinical test, the diagnosis and the treatment, and furthermore, can be applied to various medical materials, for example, a blood filtering material, a cell culture medium, a carrier of a drug delivery system, a vaccine and the like. While the utilization embodiments will be demonstrated as an example, the present inventions are not restricted to them.

If an antineurotonin-antibody or an antibody causing the peripheral nerve lesions in a blood is bound to the above-mentioned monoclonal antibody fixed to a filtering material, and through a dialytic filtration of the blood, the filtered blood is then returned to a patient again, it is possible to reduce the neurotonin antibody or the antibody causing the peripheral nerve lesions in a body. Such filtration of the blood in exchange for a whole blood dialysis provides a therapeutic method of releasing a numbness caused by the neurotonin or the antibody thereof, or the antibody causing the peripheral nerve lesions. The present invention also provides the utilization of a blood filtering material binding the antibody for this purpose.

As another embodiment, there can also be proposed the use of a cell culture medium comprising a monoclonal antibody reacting specifically with the neurotonin through an incubation with peripheral nerve and muscle system cells separated from a patient having the peripheral nerve lesions to bring about binding to the antibody concerned in the cause of the peripheral nerve lesions to thereby remove the antibody, for in vitro reproducing a cell to be a peripheral nerve.

A humanized antibody has no antigenicity to a human. Therefore, there can be proposed the utilization of such humanized antibody to be means for a treatment through binding directly to the neurotonin or other proteins, biological components and the like which are concerned in the cause of the peripheral nerve lesions including the Isaacs syndrome to thereby lose their function, or to be a DDS (Drug Delivery System) carrier to effectively transport an effective bioactive substance or a compound having the drug efficacy to a target site. In view of reaction uniformity, the human monoclonal antibody may be used particularly suitably.

A vaccine composition according to the present invention stimulates an immune response to the Isaacs syndrome or other peripheral nerve lesions in a human or other non-human animals. The vaccine may contain any polypeptide of the proteins belonging to the scope of the present invention, together with a pharmaceutically-acceptable carrier to thereby stimulate an immune response to the polypeptide, or contain a virus cell or a host cell having any of the above-mentioned nucleic acid molecules inserted therein to thereby stimulate an immune response to a polypeptide to be encoded by the inserted nucleic acid molecule.

The vaccine composition can be manufactured according to a well-known method in a vaccine manufacturing field. Such a vaccine prescription may conventionally include at least one polypeptide according to the present invention together with at least one appropriate adjuvant, for example, aluminum hydroxide, saponin, Quil A or a refined form thereof, muramyldipeptide, mineral oil, novasom or the like if it is proper under the presence of at least one pharmaceutically acceptable carrier or diluent. Examples of a preferable carrier include a liquid medium such as a physiological saline solution suitable for a vehicle to be used for introducing a peptide or a polypeptide to a patient. An additive component such as an antiseptic may be contained.

As another vaccine prescription, it may also contain a virus in which the nucleic acid molecule according to the present invention is inserted, or a host cell, or a microorganism such as a vaccinia virus, an adenovirus or a Salmonella tribe. This is for stimulation of an immune response to the polypeptide to be encoded by the inserted nucleic acid molecule.

Accordingly, another aspect of the present invention is the method using the nucleic acid molecule or polypeptide according to the present invention in the manufacture of the vaccine composition to stimulate the immune response to the Isaacs syndrome or the other peripheral nerve lesions in the human or the other animals.

For the administration of such a vaccine, a conventional route, for example, an oral or non-oral administration can be optionally utilized (for example, an intramuscular injection at an optional interval, that is, two injections every 7 to 28 days).

Animal

In order to search the neurotonin gene and the function and expression control of the neurotonin and to establish a method of diagnosing and treating peripheral nerve lesions including the Isaacs syndrome based thereon to develop a remedy therefor, a disease model animal is useful. More specifically, an animal model for peripheral nerve lesion according to the present invention is a transgenic non-human vertebrate which could be modified in the expression of a DNA encoding the neurotonin or a DNA which is homologous to or hybridized with a nucleotide sequence shown in FIG. 1 or 2 or a nucleotide sequence shown in FIG. 3 or 4, or could induce the modification.

Examples of a more useful transgenic non-human animal include a non-human vertebrate, that is, a knockout non-human vertebrate in which the expression of an inherent neurotonin gene DNA or a related DNA thereto is suppressed and a non-human vertebrate having other genes knocked out. Such other genes exclude the gene encoding the neurotonin and may be genes encoding other proteins of a peripheral nerve cell, for example, a “14-3-3” protein or the like. The type of the non-human vertebrate is not restricted. These animals can also be utilized as the model animals of the peripheral nerve lesions for the above-mentioned purpose.

As the non-human vertebrate to be used particularly preferably, it is possible to easily produce a transgenic mouse having the gene knocked out. Such a mouse can be widely utilized for the elucidation of the mechanism of the peripheral nerve lesions, in addition to the function of the gene which is knocked out, the establishment of the diagnosing and treating method, the development of chemicals, the test of a vaccine and the like. Such a transgenic mouse can be created by a well-known method of forming, in a mouse fertilized ovum, a DNA reversely transcribed to the gene to be knocked out or by a well-known antisense method of introducing an antisense RNA. In these cases, such production can be carried out through a targeting vector using a neurotonin gene obtained from a mouse genome. Furthermore, it is also possible to use embryonic stem cells, that is, ES cells.

An animal immunized with the neurotonin may be used for screening a compound having the improvement action of the Isaacs syndrome or the other peripheral nerve lesions. Such an animal is not particularly restricted to but maybe of any type evoking an immune response. For this purpose, the animals such as rabbits, sheep, mice and rats have widely been utilized.

For example, in the rabbit immunized with the human neurotonin as described above, an antibody against the human neurotonin is raised before and after six weeks and a similar symptom to the Isaacs syndrome is manifested. The rabbit having the similar symptom to the Isaacs syndrome may be used for examining the function of antibodies such as the monoclonal antibody or the effectiveness of vaccines and chemicals or screening a compound blocking a factor to be bound to the neurotonin, a compound bound to the protein to modulate the function thereof, a protein or a non-protein substance which improves the suppression of a VGKC function, a signal transduction disorder in a nerve terminal or a painful muscular convulsion, or the like.

A compound to increase or reduce the activity of an intrinsic promoter of the DNA in relation to the expression of the neurotonin gene can be screened by a method other than the method described in the column of “related protein”, the method comprising the steps of:

a) applying a compound to be tested to a non-human vertebrate or a cell derived from the non-human vertebrate, and b) selecting the compound to increase or reduce the expression of the gene which is knocked in.

### ADVANTAGE OF THE INVENTION

According to the present invention, the neurotonin supposed to be the key substance of the Isaacs syndrome has been identified and the structure thereof was determined. Therefore, it is possible to produce or prepare a monoclonal antibody thereto, a compound capable of modulating the function of the substance, an animal applicable to screening the compound and the like. It is possible to establish a testing method and a diagnosing and therapeutic method for the peripheral nerve lesions including the Isaacs syndrome by utilizing the neurotonin, the related substance thereto and the animals.

According to the present invention, the presence of the neurotonin gene is demonstrated by the isolation and identification of a cDNA thereof. By utilizing an antibody to the neurotonin or to the antibody causing the peripheral nerve lesions, a monoclonal antibody and a transgenic mouse, consequently, useful means for studies of the function of the neurotonin gene and a mechanism of the expression, a regulating function and the like are provided, and furthermore, clarification of the mechanism of a functional disorder in the peripheral nerves, an entity and the like can be carried out.

### EXAMPLE

The present invention will further be described below based on examples and the scope of the present invention is not restricted to the description.

### Example 1

Gene Cloning of an Antigen (Neurotonin) Recognized by the Serum of an Isaacs Syndrome Patient

A cDNA encoding a neurotonin was obtained by immunoscreening using the serum antibody of an Isaacs syndrome patient expressing the corresponding gene of a human genome. Total RNAs were extracted from human neuroblastoma (NB-1) strains by an acid guanidine/phenol chloroform method, and furthermore, mRNAs were separated by an affinity chromatography using oligo (dT) beads (Anal. Biochem., 162:159, 1987). By using a λ ZAP vector (STRATAGENE Co., Ltd.), a cDNA library was formed in accordance with an ordinary method. By using a picoBlue immunoscreening Kit (STRATAGENE Co., Ltd.), an immunoscreening using the serum of the patient was carried out. A positive clone (phage) thus obtained was converted into a plasmid pBluescript II SK(+) by a helper phage. The nucleotide sequence of a DNA inserted in the pBluescript II SK(+) was determined by an ABI PRISM377 DNA Sequencing System (Perkin Elmer Co., Ltd.) based on a dye terminator method (Proc. Natl. Acad. Sci. USA., 74:5463, 1977) using M13PrimerM4 and M13PrimerRV(Takara). A nucleotide sequence was determined from the 3′ terminal of the cDNA encoding an antigen (neurotonin) recognized by the serum of the Isaacs syndrome patient to elucidate a nucleotide sequence containing a Poly(A)⁺ chain (FIG. 1). The nucleotide sequence was subjected to a homology search with GenBank. As a result, a similar sequence has not been reported and it was found that the nucleotide sequence is a novel gene. The cDNA (referred to as a “short cDNA”) is constituted by 1347 nucleotides in total and encodes a novel protein comprising 386 amino acids.

Although an attempt for obtaining a protein to be encoded by the cDNA was made, the methionine initiation codon was not found. For this reason, the nucleotide sequence was used to perform PCR by a 5′-RACE (Rapid Amplification of cDNA Ends) method (Proc. Natl. Acad. Sci. USA., 85:8998-9002, 1988) through the cDNA library. Consequently, a cDNA (referred to as a “long cDNA”) corresponding to a protein consisting of 511 amino acids in total, that is, the neurotonin was obtained.

FIGS. 1 and 2 show “short cDNA” and “long cDNA” nucleotide sequences, respectively.

### Example 2

An expression of a Recombinant Protein in Escherichia Coli

A short cDNA encoding a part of a neurotonin was extracted from a cDNA clone obtained by the immunoscreening according to the example 1. The short cDNA having a recognition sequence for an EcoRI/XhoI at a terminal was inserted into a glutathione S-transferase (GST) fused protein expression vector pGEX-5×-3 to perform subcloning. The pGEX-5×-3 incorporating the short cDNA was introduced into a BL21 Escherichia coli strain by a heat shock method at 42° C. for 45 seconds so that a BL21/short cDNA-GST gene/pGEX-5×-3 was obtained. The BL21 was cultured in an LB culture medium containing 0.1 mg/mL ampicillin, and 0.1 mM isopropylthio-β-D-galactoside (IPTG) was added, and furthermore, the cultivation was carried out at 37° C. for two hours to induce the expression of the aforesaid fused protein. The BL21 collected by a centrifugation was washed with PBS (Phosphate buffered saline) and was then subjected to 1 mg/mL lysozyme digestion, and was solubilized with 0.1% TritonX-100. A BL21 derived protein suspension containing the solubilized GST fused protein was applied to a glutathione Sepharose 4B (GS4B) column and was then washed with PBS to purify a target GST-short neurotonin fused protein by 50 mM reduced form glutathione/PBS. Furthermore, a molecular weight, a homogeneity, a subunit structure and the like were examined by SDS-electrophoresis (SDS-PAGE).

An Expression of a Protein by a Full Length cDNA Recombinant Encoding a Neurotonin in Escherichia Coli

A neurotonin cDNA was obtained by adding two molecules of an influenza hemagglutinin (HA)-tag to the 3′ terminal of a full length cDNA having the EcoRI/XhoI recognition sequence at a terminal which was obtained in the Example 1, was inserted into a glutathione S-transferase (GST) fused protein expression vector pGEX-5×-1 as an expression vector and was subjected to subcloning. The pGEX-5×-1 incorporating the cDNA was introduced into a BL21 Escherichia coli strain by a heat shock method at 42° C. for 45 seconds so that a BL21/long cDNA-GST gene/pGEX-5×-1 was obtained. The BL21 was cultured in an LB culture medium containing 0.1 mg/mL ampicillin and 0.1 mM isopropylthio-β-D-galactoside (IPTG) was added. Furthermore, the cultivation was carried out at 30° C. for three hours to induce the expression of a fused protein (GST-neurotonin-HAHA) attaching GST and HA to N and C terminals respectively. The BL21 collected by a centrifugation was washed with PBS and was then subjected to 1 mg/mL lysozyme digestion, and was solubilized with 0.1% Triton X-100. A BL21 derived protein suspension containing the solubilized GST fused protein was applied to a glutathione sepharose 4B (GS4B) column and was then washed with PBS to purify a target GST neurotonin-HAHA fused protein by 50 mM reduced form glutathione/Tris-HCl (pH 8.0).

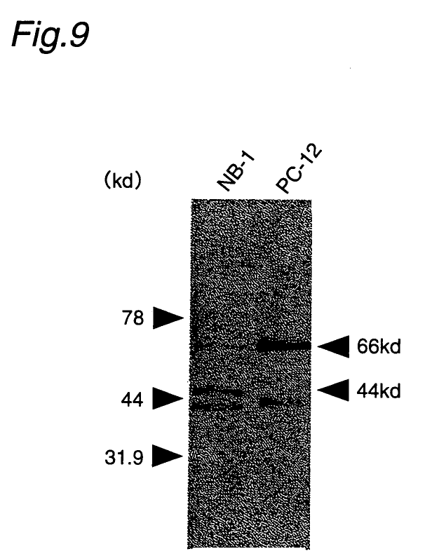

For the confirmation of the expression, a fraction eluted with 50 mM reduced form glutathione was diluted in 1/200 and 0.1/2,000 with PBS and was then treated with 25 mMTris-HCl (pH 6.8), containing 0.25% SDS, 0.05% mercaptoethanol and 0.1% glycerol, and was there after applied to 8% SDS-PAGE. After the SDS-PAGE, the fused protein (GST-neurotonin-HAHA) was transferred to a nylon membrane by an electroblotting method. The nylon membrane was subjected to blocking at a room temperature for 60 minutes with PBS containing 5% skimmed milk and then an immunoreaction was effected at a room temperature for 60 minutes with an anti-HA monoclonal antibody (Boehringer Mannheim Co., Ltd.) diluted in 1/400 with PBS containing 0.5% skimmed milk. After the reaction, washing was conducted with 0.1% Tween20/PBS and another immunoreaction was carried out at a room temperature for 60 minutes by using a horseradish peroxidase conjugated -antimouse-immunoglobulin G (anti-mouce IgG-HRP) as a secondary antibody, and washing was then conducted with 0.1% Tween 20/PBS to detect a target antigen through manifesting an HRP activity. ECL (Amersham Co., Ltd.) was used for the detection of the HRP activity (Clin. Chem., 25:1531, 1979). A result is shown in FIG. 9. The molecular weight of the neurotonin was estimated to be approximately 66 KDa based on the molecular weight size of the GST-neurotonin-HAHA fused protein.

An Expression of a Protein In Vitro by a Full Length cDNA Recombinant

A neurotonin cDNA (FIG. 2) was modified at its terminal by a restriction enzyme EcoRI and was then inserted in a pBluescript IIKS vector. Thereafter, by using the pBluescript (lag) and TNT-coupled Translation System (Promega Co., Ltd.), a neurotonin was expressed as a (³⁵S) labeled entity in vitro by an in-vitro translation method. The (³⁵S) labeled protein was applied to 10% SDS-PAGE and a radioactivity was detected by an imaging analyzer (BAS2000, Fujix). It was observed that the molecular weight of the neurotonin translated via the neurotonin cDNA in vitro based on the SDS-PAGE is approximately 66 KDa.

### Example 3

A Confirmation of an Expression of a Neurotonin Gene by a Northern Blotting Method

mRNAs were isolated from an NB-1 cell, an A549 cell strain, a Jurkat cell strain and an HeLa cell strain by the conventional method. 1 μg of the mRNAs was separated by 1% agarose gel electrophoresis and was transferred to a nylon membrane by a contact blotting method. The nylon membrane was treated at 80° C. for two hours and then a prehybridization was carried out at 0.42° C. for two hours in a Denhardt's solution. Next, a neurotonin cDNA using a neurotonin cDNA radioactive-labeled with ³²P as a probe was hybridized with the mRNA at 42° C. for 12 hours. The nylon membrane thus obtained after the reaction was washed with 300 mM NaCl and 30 mM sodium citrate and was then washed again at 50° C. by using 15 mM NaCl and 1.5 mM sodium citrate. A target mRNA was detected by photosensitizing an X-ray film. From an autoradiograph thus obtained, it was observed that a neurotonin gene was strongly expressed in the NB-1 cell of a nerve cell.

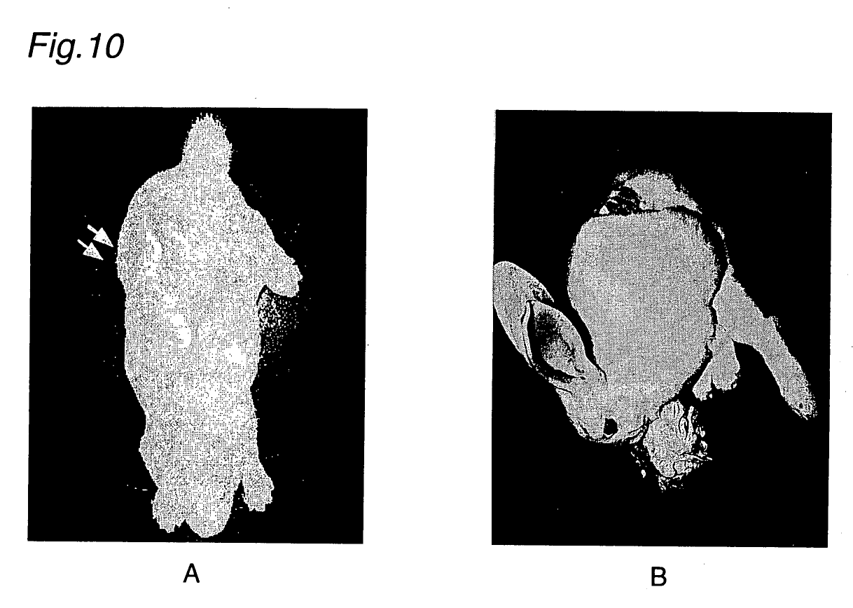

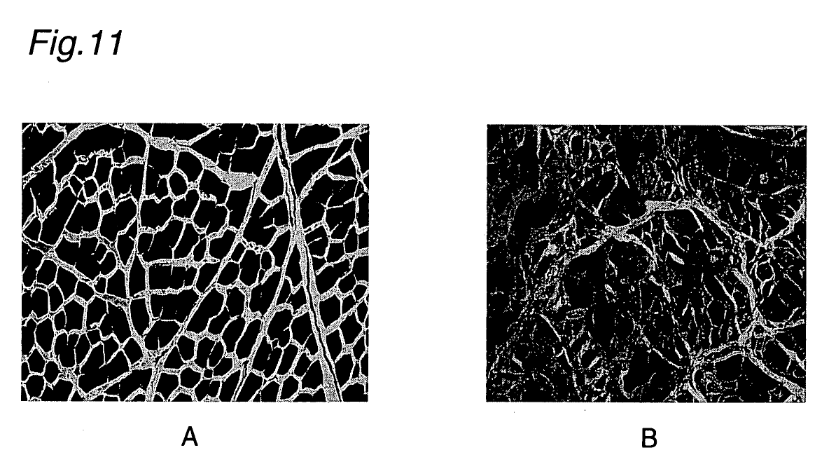

An immunizationa of a rabbit with a neurotonin (FIGS. 10 and 11)

By using a neurotonin comprising 511 amino acids in total, a fused protein thereof with GST was produced and was then used as an antigen to immunize three rabbits together with an adjuvant in accordance with an ordinary method. After six weeks, it was observed that peripheral nerve lesions having similar symptoms to the Isaacs syndrome, for example, a numbness on hind limbs, a muscle rigidity, dysbasia based thereon and the like were induced to all the three rabbits (FIG. 10). A muscle obtained from the rabbit immunized with the neurotonin was subjected to HE staining and was observed microscopically. As compared with a normal muscular tissue, consequently, the size of a muscular cell was nonuniform and the denaturation of a nerve cell was also observed (FIG. 11).

Next, a human neurotonin was divided into three sections, that is, a C terminal region portion, an N terminal region portion and their middle portion. They were separately immunized to five rabbits. Consequently, the above-mentioned symptoms were induced to the rabbit immunized to the C terminal portion of the neurotonin. In the case in which other portions were immunized, such a result was not obtained in the rabbits.

A serum obtained from the rabbit immunized with the neurotonin was used as an anti-neurotonin antiserum in the following experiments. A confirmation of an expression of a neurotonin gene in various cells by a Western blotting method

By using a GST-short neurotonin fused protein (positive control) and an NB-1 nerve cell which were prepared in the Example 1 and an HEK (human embryonic kidney)—293T, the expression state of a neurotonin gene was confirmed by a Western blotting method.

First of all, cytolysates solubilized with 1% NP-40 were prepared from various cells to be samples. Each of the cytolysates was treated with25 mMTris-HCl (pH 6.8), containing 0.25% SDS, 0.05% mercaptoethanol and 0.1% glycerol and was separated through 8% SDS-PAGE. After the SDS-PAGE, proteins derived from various cells were transferred to a nitrocellulose (NC) membrane by an electroblotting method. On the NC membrane, the anti-neurotonin antiserum was diluted in 1/1,000 with TBS (Tris Buffered Saline) which was added by 2.0 mg/mL GST-short neurotonin fused protein and 5% skimmed milk and allowed to give rise to an immunoreaction at a room temperature for 60 minutes. Moreover, an experiment for allowing the same antibody solution to react with the NC membrane only as a negative control or an experiment for exchanging the GST-short neurotonin fused protein of the antibody solution for only GST was carried out at the same time. The NC membrane obtained after the reaction was washed with 0.1% Tween 20/TBS and then was subjected to an immunoreaction at a room temperature for 60 minutes with an HRP labeling anti-rabbit IgG antibody as a secondary antibody, and was washed with 0.1% Tween 20/TBS to detect an HRP activity so that a target antigen was detected. For the detection of the HRP activity, ECL (AmershamCo., Ltd.) was used (Clin. Chem., 25:1531, 1979). As a result, it was observed that the neurotonin is produced in an NB-1 cell.

### Example 4

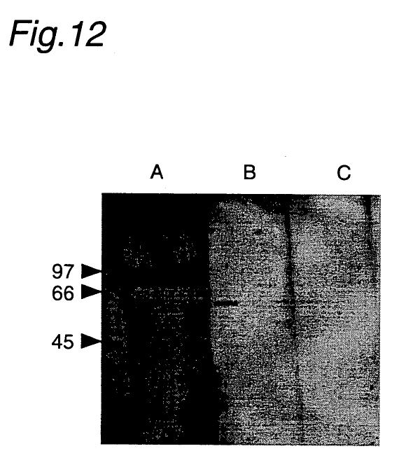

Detection of an Anti-Human Neurotonin Antibody in a Serum of a Patient (FIG. 12)

By using the GST-short neurotonin-fused protein prepared as described above as an antigen, an anti-neurotonin antibody presenting in a human serum was detected by a Western blotting method.

First of all, a GST-short neurotonin-fused protein (100 ng/lane) was transferred to an NC membrane by SDS-PAGE electrophoresis. For a primary antibody were used sera obtained from (five) Isaacs syndrome patients, (thirty) patients having other peripheral nerve lesions other than the Isaacs syndrome who complained of the painful numbness of hands and legs as a complication of diabetes or the like, and (eight) people in normal health. In order to examine the presence of an anti-neurotonin antibody in the serum, a serum sample diluted in 1/1,000 with TBS was applied to the NC membrane to give rise to an immunoreaction at a room temperature for 60 minutes. The NC membrane was washed with 0.1% Tween 20/TBS and was then subjected to an immunoreaction at a room temperature for 60 minutes by using an HRP labeled anti-human IgG antibody as a secondary antibody. The NC membrane was washed with 0.1% Tween 20/TBS to detect an HRP activity. Thus, a human IgG reacting to a target antigen was detected. The HRP activity was measured by the above-mentioned method.

As a result, the anti-neurotonin antibody was detected from four of the five Isaacs syndrome patients and seven of the thirty patients having the peripheral nerve lesions other than the Isaacs syndrome, and was not detected from the (eight) people in normal health. FIG. 11 shows a part of the results.

Referring to the sera of the patients from which the antibody was detected, an antibody directed to the C terminal region of the neurotonin was detected from the four positive Isaacs syndrome patients and was similarly detected from the seven positive patients having the peripheral nerve lesions other than the Isaacs syndrome, and furthermore, an antibody directed to the N terminal portion of the neurotonin was detected from two other people.

### Example 5

Detection of a Neurotonin Encoding cDNA by Hybridization Cloning of a Mouse Genome

Asa library, Mouse Genomic Library (Clontech Co., Ltd.) derived from a mouse liver was used and the full length sequence of the human neurotonin cDNA was employed for a probe. The probe was labeled with a radioisotope (³²P) by using Bca BEST Labeling Kit (Takara Co., Ltd.). Phage DNAs per plate were transferred to two sheets of GeneScreen Plus (Du Pont Co., Ltd.), and a hybridization was carried out overnight with the labeled probe in a reaction solution (1M NaCl, 1% SDS (Sodium Dodecyl Sulfate), 10% Dextran, 100 g/ml salmon sperm DNA) After the hybridization, the filter was washed twice at a room temperature with 2×SSC (an abbreviation of Standard Saline Citrate which implies 0.15M NaCl and 0.015M sodium citrate pH 7.2), twice at 65° C. with 2×SSC/1% SDS, and twice at the room temperature with 0.1×SSC, and a positive plague was then detected by autoradiography. Under the same conditions, second and third screening operations were carried out to detect and isolate the positive plague by using an imaging analyzer (Fujix Co., Ltd.).

The phage DNA used in the analysis was purified by using Wizard™ Lambda Preps DNA Purification System (Promega Co., Ltd.). As a result, nine positive clones were obtained.

Southern Blot Analysis

In order to examine that nine positive clones obtained by hybridization cloning contain a region including an ATG equivalent to the methionine initiation codon, the following analysis was conducted.

1 ng of a genome phage DNA thus purified was digested with a restriction enzyme SacI and was separated by 0.5% agarose electrophoresis. A gel is subjected to ethidium bromide staining and the genome DNA was confirmed to be digested, and a DNA in the gel was then transferred onto a filter overnight with an alkaline buffer (0.4M NaOH, 0.6M NaCl) by a blotting stone method. The filter neutralized with 50 MM NaOH and 1M Tris-HCl (pH 8.0) was air-dried and was then immersed in a prehybridization solution (1% SDS, 1M NaCl, 10% Dextran), and was thereafter subjected to the prehybridization at 65° C. for 30 minutes.

Next, a probe to specifically recognize a region containing an ATG equivalent to the methionine initiation codon of the neurotonin cDNA and a flanking sequence thereof was labeled with a radioisotope (³²P) by using Bca BEST Labeling Kit (Takara Co., Ltd.). The probe and 100 g/ml salmon sperm DNA were added to the aforesaid prehybridization solution to carry out a hybridization overnight at 65° C. After the hybridization was ended, the filter was washed twice at a room temperature with 2×SSC, twice at 65° C. with 2×SSC/1% SDS, and twice at the room temperature with 0.1×SSC. Then, the filter was photosensitized into an imaging plate to detect and isolate positive plagues by using an imaging analyzer (Fujix Co., Ltd.).

As a result, it was found that a 5′ region containing ATG corresponding to the methionine initiation codon is present in three of the nine clones. More specifically, approximately 2 kb of band was detected from one clone and approximately 7 kb of band was detected from two clones. These three clones are found to be genome DNAs containing a 5′ upstream region.

### Example 6

Isolation of a mouse cDNA

It is possible to obtain a cDNA encoding a mouse neurotonin by the hybridization cloning of a mouse genome in the following manner.

A mouse embryo derived Mouse Embryo Lambda cDNA Library (STRATAGENE Co., Ltd.) was used as a library and the full length sequence of the human neurotonin cDNA employed for a probe. The probe was labeled by using Bca BEST Labeling Kit (Takara Co., Ltd.) with a radioisotope (³²P) Phage DNAs per plate were transferred to two sheets of GeneScreen Plus (Du Pont Co., Ltd.), and was allowed to react with the labeled probe at 65° C. for eight hours in a reaction solution (1M NaCl, 1% SDS, 10% Dextran, 100 g/ml salmon sperm DNA). The filter was washed twice at a room temperature with 2×SSC, twice at 65° C. with 2×SSC/1% SDS, and twice at the room temperature with 0.1×SSC, and a positive plague was then detected by autoradiography. Under the same conditions, second and third screening operations were carried out to detect and isolate positive plagues by using an imaging analyzer (Fujix Co., Ltd.).

The phage DNA used in the above analysis was purified by using Wizard™ Lambda Preps DNA Purification System (Promega Co., Ltd.). As a result, one positive clone was obtained.

A genome phage DNA 1 thus purified was cleaved by restriction enzymes EcoRI and XhoI and an insert was then cut out. The insert was further subjected to subcloning, at EcoRI and XhoI sites of a cloning vector, pBluescriptII. Consequently, a cDNA encoding a mouse neurotonin could be obtained. In the analysis of the nucleotide sequence of the cDNA, the sequence was determined by an ABIPRISM377 DNA Sequencing System (Perkin Elmer Co., Ltd.).

The nucleotide sequence thus analyzed was converted into an amino acid sequence. Consequently, it was found that said sequence contains the methionine initiation codon and is homologous with the nucleotide sequence of a human neurotonin by 85% or more.

1. A peripheral nerve cell protein having the following properties (which is hereinafter referred to as a “neurotonin”),
wherein the protein
a) is found in peripheral nerve cells of Isaacs syndrome patients or other patients having peripheral nerve lesions,
b) reacts with an antibody found in a blood of the patients,
c) causes a similar symptom to an Isaacs syndrome group with an immunization to a rabbit, and
d) has a molecular weight of approximately 66 kDa according to SDS-PAGE.

2. A cDNA encoding a neurotonin.

3. A DNA encoding a neurotonin or including all nucleotide sequences shown in FIG. 1 or 2.

4. A nucleic acid molecule encoding a neurotonin, including at least one nucleotide sequence substantially corresponding to a whole or part of the nucleotide sequences shown in FIG. 1 or 2, or including a sequence which is substantially homologous with any of the sequences or is hybridized with any of the sequences.

5. A mouse cDNA encoding a mouse neurotonin.

6. A DNA encoding a mouse neurotonin or including all nucleotide sequences shown in FIG. 3 or 4.

7. A nucleic acid molecule encoding a mouse neurotonin, including at least one nucleotide sequence substantially corresponding to a whole or part of the nucleotide sequences shown in FIG. 3 or 4, or including a sequence encoding the mouse neurotonin which is substantially homologous with any of the sequences or is hybridized with any of the sequences.

8. An antisense RNA or an antisense DNA to the cDNA according to claim 2 or 5 or the nucleic acid molecule according to claim 3, 4, 6 or 7.

9. A ribozyme recognizing and cleaving the cDNA according to claim 2 or 5, the nucleic acid molecule according to claim 3, 4, 6 or 7, or an RNA transcribed from a part thereof.

10. An expression vector, a cloning vector or a cosmid containing the nucleic acid molecule according to any of claims 2 to 8.

11. A transformant retaining the vector or the cosmid according to claim 10.

12. A prokaryotic cell, a eukaryotic cell or a variant cell which contains the nucleic acid molecule according to any of claims 2 to 8.

13. A neurotonin having an amino acid sequence shown in FIG. 5 or 6.

14. A protein defined in the following a) or b) which has an activity to bind to an antibody found in a blood of the patient:
a) a polypeptide such as a polypeptide having an amino acid sequence shown in FIG. 5 or 6, a polypeptide having an amino acid sequence in which at least one amino acid is deleted, substituted or added and/or inserted in the amino acid sequence shown in FIG. 5 or 6, a synthetic polypeptide including an amino acid sequence constituting an antigenic portion of a neurotonin, functionally equivalent variants thereof, or a fused polypeptide further including the aforesaid amino acid sequences; and
b) a protein encoded by a DNA to be hybridizable with the DNA having the nucleotide sequence according to any of claims 2 to 4.

15. A protein defined in the following a) or b) which can bind to a “14-3-3” protein existing in a peripheral nerve cell:
a) a polypeptide such as a polypeptide having an amino acid sequence shown in FIG. 5 or 6, a polypeptide having an amino acid sequence in which at least one amino acid is deleted, substituted or added and/or inserted in the amino acid sequence shown in FIG. 5 or 6, a synthetic polypeptide including an amino acid sequence constituting an antigenic portion of a neurotonin, functionally equivalent variants thereof, or a fused polypeptide further including the aforesaid amino acid sequences; and
b) a protein encoded by a DNA to be hybridizable with the DNA having the nucleotide sequence according to any of claims 2 to 4.

16. An immunologically active domain of the protein according to any of claims 13 to 15 or a fragment having the domain.

17. A polypeptide such as a polypeptide having an amino acid sequence shown in FIG. 7 or 8, a polypeptide having an amino acid sequence in which at least one amino acid is deleted, substituted or added and/or inserted in the amino acid sequence shown in FIG. 7 or 8, a synthetic polypeptide including an amino acid sequence constituting an antigenic portion of a neurotonin, functionally equivalent variants thereof, or a fused polypeptide further including the aforesaid amino acid sequences.

18. A reagent comprising the protein according to any of claims 13 to 17 for an immunological analysis to assay an antibody recognizing the protein.

19. The reagent for an immunological analysis according to claim 18 for use to assess a diagnosis or treatment effect of an Isaacs syndrome or other peripheral nerve lesions.

20. A reagent comprising an antibody to react with the protein according to any of claims 13 to 17 for an immunological analysis to assay the protein.

21. The reagent for an immunological analysis according to claim 20 for use to assess a diagnosis or treatment effect of an Isaacs syndrome or other peripheral nerve lesions.

22. The reagent for an immunological analysis according to claim 21, wherein the protein according to any of claims 13 to 17 to be analyzed is present in peripheral nerve cells.

23. A neurotonin detecting method of analyzing an antibody which is present in a body fluid of a subject and reacts with the protein according to any of claims 13 to 17.

24. A method of screening a ligand to bind to the protein according to any of claims 13 to 17, comprising the steps of:
a) causing a candidate compound of the ligand to come in contact with the protein according to any of claims 13 to 17; and
b) selecting a candidate compound having a binding activity to the protein.

25. An assay method of the protein according to any of claims 13 to 17 on the basis of an affinity of an antiligand and the ligand obtainable by the method according to claim 24 by using, as the antiligand, the protein.

26. A screening method of a compound inhibiting a binding of the protein according to any of claims 13 to 17 to the ligand, comprising the steps of:
a) causing the protein according to any of claims 13 to 17 to come in contact with the ligand under the presence of a candidate compound; and
b) selecting a candidate compound having an activity to inhibit the binding of the protein to the ligand.

27. A compound which can be obtained by the screening method according to claim 26.

28. A neurotonin inhibitor comprising a compound which can be obtained by the screening method according to claim 26.

29. A medicine for treating peripheral nerve lesions, comprising a compound which can be obtained by the screening method according to claim 26.

30. An antibody binding to the protein according to any of claims 13 to 17.

31. The antibody according to claim 30, wherein the antibody is a monoclonal antibody.

32. A monoclonal antibody against an antibody causing peripheral nerve lesions.

33. A method of detecting or separating a cell expressing the protein according to any of claims 13 to 17 by setting, as a marker, the protein or an expression of a gene encoding the protein.

34. The method according to claim 33, wherein the cell is a peripheral nerve cell.

35. A reagent for detecting or separating a cell expressing the protein according to any of claims 13 to 17, comprising the antibody according to any of claims 30 to 32.

36. A reagent for detecting the DNA according to claims 2 to 7 or an RNA transcribed therefrom, comprising a nucleic acid molecule having a nucleotide sequence which can hybridize with the DNA or a part thereof, or the RNA.

37. A transgenic non-human vertebrate in which an expression of the DNA according to claim 3 or 6 is modified or inducible the modification.

38. The animal according to claim 37 which is a peripheral nerve lesion model animal.

39. The knockout non-human vertebrate according to claim 37, wherein an expression of the intrinsic DNA according to claim 3 or 6 is suppressed.

40. The non-human vertebrate according to claim 37, wherein another gene is knocked out.

41. A cell derived from the non-human vertebrate according to any of claims 37 to 40.

42. A method of screening a compound to increase or reduce an activity of an intrinsic promoter of the DNA according to claim 3 or 6, comprising the steps of:
a) detecting an expression of a gene bound to a downstream region of the intrinsic promoter of the DNA according to claim 3 or 6 under the presence of a compound to be tested; and
b) selecting the compound to increase or reduce the expression.

43. A method of screening a compound to increase or reduce an activity of an intrinsic promoter of the DNA according to claim 3 or 6, comprising the steps of:
a) applying a compound to be tested to the non-human vertebrate according to claims 37 to 40 or a cell derived from the vertebrate; and
b) selecting the compound to increase or reduce an expression of a gene which is knocked in.

44. An animal immunized with a neurotonin which is to be used for studies of an Isaacs syndrome or other peripheral nerve lesions and for screening of a substance having an improvement action of the Isaacs syndrome or other peripheral nerve lesions.

45. A rabbit immunized with a neurotonin for screening a protein or a non-protein substance which improves a suppressed VGKC function, a signal transduction disorder in a nerve terminal or a painful muscular convulsion.

46. A diagnostic marker for detecting a specific binding to the monoclonal antibody by using, as a marker, a neurotonin, a related protein thereto or other antibodies causing peripheral nerve lesions, thereby measuring a level of a neurotonin, a related protein thereto or an antibody causing peripheral nerve lesions in a sample obtained from a subject in order to diagnose an Isaacs syndrome or other peripheral nerve lesions.

47. An examination method of offering data for diagnosing an Isaacs syndrome or other peripheral nerve lesions, wherein a level of a neurotonin or an antibody causing peripheral nerve lesions in a sample obtained from a subject is measured.

48. A blood filtering material for use in eliminating a numbness caused by the presence of a neurotonin or an antibody causative peripheral nerve lesions to carry out a treatment which allows a neurotonin antibody or an antibody causing peripheral nerve lesions in a blood to bind to the monoclonal antibody fixed onto the filtering material through a dialytic filtration of the blood, thereby reducing the neurotonin antibody or the antibody causative the peripheral nerve lesions in a body.

49. A vaccine composition for stimulating an immune response to an Isaacs syndrome or other peripheral nerve lesions in a human being or other animals which comprises at least one polypeptide according to any of claims 13 to 17 together with a pharmaceutically acceptable carrier and stimulates an immune response against the polypeptide, or which comprises a virus cell or a host cell having the nucleic acid molecule according to any of claims 2 to 8 which is inserted in the cell and stimulates an immune response against a polypeptide encoded by the inserted nucleic acid molecule.

50. A cell culture medium comprising the monoclonal antibody according to claim 31 or 32 for in vitro reproducing a cell to be a peripheral nerve by removing an antibody causative peripheral nerve lesions through an incubation with peripheral nerve and muscle system cells separated from a patient having peripheral nerve lesions, to thereby cause a specific binding of the causative antibody to the monoclonal antibody.


SEQUENCE LISTING (4 sequences)

SEQ ID NO: 1 - DNA, 1347 bp, human
  Short cDNA for neurotonin
tgacagaagggagaagaaagaaatgaagcgagttttcagtattccaagcataagagccagcaagatacattccctcaagtgtccagaatttccaattacagacgacaaagtagcactgattcgaattcagaattgtcaaatgaagaattaaggcaatgtcttaatgaaactttagaggaggtagaaatgttaaaaactgaacttgaggcatctcaaagacaactcagaggtaaagaggaagcattgaaaattcttcaaagcatggcaatactgggcaaagccacaagtcatacgcaggcagtgcttcaaaaaactatggaacaaaacagatccttggagaaggaaataaatgccttgcagtgggaaatagaatttgatcataatagatttaaaaatatagaggaatcttggatccaaaaatatgacaggctaaactgtgaaaatgcagtcctcaaagagaatttgaaagtgaaaacagaagaaattaaaatgctgaagtctgacaatgcagttttgaatcaacggtatttggaggccctcgccatgcttgatatcaaacagcagaagatggctcaggaaaacatgtgctgtgataaaagtggctttgcagaggcttcaggtcttgagcttgcggtcctcggagcctgcctttgtcatgggcccggagggaacccctgttcttgtgccagaatggcagcatccactcggaaactgcttcttcagctcaaacaagagttggaaattttgcagaagagtaaagaagaagcttacgtgatggcagatgctttcagaattgcatttgagcaacaattaatgagaaaaaatgaccaggcactacaattgacacaaatggataaaatgcataaaaaagcaacaaaatggatgaattggaagcaccttaaagaggatggatttccatcaccaaggagtaagaagaccttcgggcagagactgttgggtatgctcccttcagaaaacagttctaagaggatggaagaccaggacagtcctcaagaggtccttaagatgctcatagatttgcttaatgataaagaagaagctttggctcatcaaagaaaagttagctacatgcttgctcgggcattggaagacaaagacactgcttcaaacgagaataaagaaaaaaatcctataaaagagaatttccctttcaacaacccctggcgtaagacttcagaattctctgttttgggtgatcctatacattcaagtgtctgcattttaaattctgtgggctgcatttgttcaatccagcactctcaaatagatccaaactatagaactcttaaaagatcccattctttgccatcaagtatcatattttaa

SEQ ID NO: 2 - DNA, 2738 bp, human
  Long cDNA for neurotonin
gagctggagtccgggctccggtcccctccacggaccttgagagggtacccggggtcagacctggcagacagcccattttttcttatgataaagacggcatttggctcatgagcaaggtggcaagatcatcaagtgagtcagacatgcagctctgggaaacagaagaggatgacatgacagaaggtgatttagggtatggcctcggaaggaaacctggtgggatttatgaaatagaattttcacataggtctagaaaaagatcagatggaaagaactttagccctcctccatttccgagaaagggagaagaaagaaatgaagcgagttttcagtattccaagcataagagccagcaagatacattccctcaagtgtccagaatttccaattacagacgacaaagtagcactgtagattcgaattcagaattgtcaaatgaagaattaaggcaatgtcttaatgaaactttagaggaggtagaaatgttaaaaactgaacttgaggcatctcaaagacaactcagaggtaaagaggaagcattgaaaattcttcaaagcatggcaatactgggcaaagccacaagtcatacgcaggcagtgcttcaaaaaactatggaacaaaacagatccttggagaaggaaataaatgccttgcagtgggaaatagaatttgatcataatagatttaaaaatatagaggaatcttggatccaaaaatatgacaggctaaactgtgaaaatgcagtcctcaaagagaatttgaaagtgaaaacagaagaaattaaaatgctgaagtctgacaatgcagttttgaatcaacggtatttggaggccctcgccatgcttgatatcaaacagcagaagatggctcaggaaaacatgtgctgtgataaaagtggctttgcagaggcttcaggtcttgagcttgcggtcctcggagcctgcctttgtcatgggcccggagggaacccctgttcttgtgccagaatggcagcatccactcggaaactgcttcttcagctcaaacaagagttggaaattttgcagaagagtaaagaagaagcttacgtgatggcagatgctttcagaattgcatttgagcaacaattaatgagaaaaaatgaccaggcactacaattgacacaaatggataaaatgcataaaaaagcaacaaaatggatgaattggaagcaccttaaagaggatggatttccatcaccaaggagtaagaagaccttcgggcagagactgttgggtatgctcccttcagaaaacagttctaagaggatggaagaccaggacagtcctcaagaggtccttaagatgctcatagatttgcttaatgataaagaagaagctttggctcatcaaagaaaagttagctacatgcttgctcgggcattggaagacaaagacactgcttcaaacgagaataaagaaaaaaatcctataaaagagaatttccctttcaacaacccctggcgtaagacttcagaattctctgttttgggtgatcctatacattcaagtgtctgcattttaaattctgtgggctgcatttgttcaatccagcactctcaaatagatccaaactatagaactcttaaaagatcccattctttgccatcaagtatcatattttaaagacaagccagttgaaattggaactgagagttgtttatatcgagatactttgaaaaatgattttgtaaattttgctgcatcttgagaagttgtatgtttcctaggtatttctaaattttaggaggtcacttaaatcaaatattttctacattttctttttcttcttttgagatggagtcttgctctgtcacccaggttggagtgcagtggtgtgatctcggttcactgcaacctccgcctcccggttcaggcaattctcctgcctcagcctcccgagtgactcggattacaggcatgtgccatctctactaaaaatacaaaaaattggctgggcctagtggcgcatgcctgtaatcccagctgcttgggaggctgaggtaggagagtcgcttgagcctgggaggcgggggttgtggtgagccgagactgtgtcattgcactccagcctgggcgacaagagcgaaactctgtctcaaaaaaaaaaaaaaaacacattacagtgtttgccaacatcctaaaactaaaaaataatgatttttgggaaaaacaaaaaattatttaactattttgattgagtatagggatcttagcttggctaaatcaccaatatagcaatgtatcttaatgctctaaaaagtaacaatacctaggactgactgaatgttaatttctggaattcatttatcttgttttatttgccttgttcagtatttttcatttaccatatgaagtttagaaataacaatttccttaagttaacttcaataagacagttttgatggtgggaccttggcagaagtatattgagtcagtgccggctgcgttcatggcttcttgtcagggtagtaacattttgcaaccaaatctgcacagatttgtagccaagtctacaaagtgcttaacagctcttttcattgcatcctgtgagatggttgtgatgatccaggaaatggggtctcaggaatctgaagaagctgcctgagccaaggtgatttctaggagtttttttgtttgtggtatgtgaattggggtgtgtgggggtggggtgtgtgtgtagacagggtcccactgtgttgctcaggctggtgccaaact

SEQ ID NO: 3 - DNA, 1326 bp, mouse
  unsure 917,919,922,924: Short cDNA for neurotonin
tgaccgaagggagaagaaagaagcgaaaccagtttccagtattccaggaggaagggctttcaagatacaagcgctgagggataccgagcatccagactgagcagcacagattctaactcagaattgtcggatgagcagttaaggcgacgtcttcatgaagccttagaggacgtagagattttaaaaacggaacttgaagcgtctcaaagacaacttgaaggtaaagaggaagcattgaaaatcctccaaagcatggcaatgcttggcaaagccacaagccacacacagacaatgcttcaaaaaactatagaacaaaagagatctctggagaaggaaataaatgccttgcagtgggaaatggaatttgatcaggatagatttaaaaatatagaagaatcttggatccagaaatgtgacaggctaaactgtgacaatgcagtcctcagagagaatctgaagttgagaacagaggaaataaagatgctaaagtctaagaatgctgttttgaatcagcggtacttggaggccctcgccatgcttgatatcaaggagcagaagatgggtcaggaggagagtggctttacagatgtatcaggtctcgagcttgcagtccttggagcctgcctgtgtcatggtcctggagggagcccctgttcttgtgccaaaatggcagcatccactcggaaactggttcttcagctcagacatgagttggaaactctgcagaagagtaaggaagaggcgcacataacggcagatgcattcaggattgcttttgagcaacagttaatgaggaaaaatgagcaggcactgagactggctggaggggacctgtgtaaaaaagcggcaaccgtggatcaacagacaacacccaggcagacgatggatatccggcacaaaggagaaagaagaccttntnggntnagattactggggatactcccttcggaaaacagctccaagggcgctgaagaccaagacaatatgcaagaggtctttaagatgctggtagatttgttgaatgacaaagaagaagcccttgcacatcagagaaaggttagttacatgctcgctcgggcgctggaagacaaagacacggcctcagaaaggaataaagaaaaaatccccatgagccagaccttcccattcaaaacggcctggcacgacgcttcagagctctgtggtctgcgtgatccagtacagtcaaaccatgtttctgaacccatggcttgcatctgttcaatacagcatcctccaaaagtttcagactgcccaagaactcttaaaagatcctgttctttgccatcaactttattttacaagtaa

SEQ ID NO: 4 - DNA, 1559 bp, mouse
  Long cDNA for neurotonin
tgagggaggaggctgagtgtccagcagctccctgggaccgacatttggctaatgagcaaagtgccgaggtcatcgagcgaggcagaggacatctgggaaacagaggatgacatgaccgaaggtgacctaggctatggcctcggaagaaaacccggtggtatttatgaagttccgtgttcgattacatctaagaaaaggtcagatggaaagaactcaagccctcctccgtttccaagaaagggagaagaaagaagcgaaaccagtttccagtattccaggaggaagggctttcaagatacaagcgctgagggataccgagcatccagactgagcagcacagattctaactcagaattgtcggatgagcagttaaggcgacgtcttcatgaagccttagaggacgtagagattttaaaaacggaacttgaagcgtctcaaagacaacttgaaggtaaagaggaagcattgaaaatcctccaaagcatggcaatgcttggcaaagccacaagccacacacagacaatgcttcaaaaaactatagaacaaaagagatctctggagaaggaaataaatgccttgcagtgggaaatggaatttgatcaggatagatttaaaaatatagaagaatcttggatccagaaatgtgacaggctaaactgtgacaatgcagtcctcagagagaatctgaagttgagaacagaggaaataaagatgctaaagtctaagaatgctgttttgaatcagcggtacttggaggccctcgccatgcttgatatcaaggagcagaagatgggtcaggaggagagtggctttacagatgtatcaggtctcgagcttgcagtccttggagcctgcctgtgtcatggtcctggagggagcccctgttcttgtgccaaaatggcagcatccactcggaaactggttcttcagctcagacatgagttggaaactctgcagaagagtaaggaagaggcgcacataacggcagatgcattcaggattgcttttgagcaacagttaatgaggaaaaatgagcaggcactgagactggctggaggggacctgtgtaaaaaagcggcaaccgtggatcaacagacaacacccaggcagacgatggatatccggctacaaaggagaaagaagaccttagggcaaagattactggggatactcccttcggaaaacagctccaagggcgctgaagaccaagacaatatgcaagaggtctttaagatgctggtagatttgttgaatgacaaagaagaagcccttgcacatcagagaaaggttagttacatgctcgctcgggcgctggaagacaaagacacggcctcagaaaggaataaagaaaaaatccccatgagccagaccttcccattcaaaacggcctggcacgacgcttcagagctctgtggtctgcgtgatccagtacagtcaaaccatgtttctgaacccatggcttgcatctgttcaatacagcatcctccaaaagtttcagactgcccaagaactcttaaaagatcctgttctttgccatcaactttattttacaagtaaac